(19) United States
(12) Patent Application Publication
(10) Pub. No.: US 2014/0050728 A1
(43) Pub. Date: Feb. 20, 2014

(54) METHODS AND COMPOSITIONS FOR INHIBITING CYCLOPHILIN D FOR THE TREATMENT AND PREVENTION OF OBESITY AND KIDNEY INDICATIONS

(71) Applicant: Babu Padanilam, Omaha, NE (US)

(72) Inventor: Babu Padanilam, Omaha, NE (US)

(73) Assignee: BOARD OF REGENTS OF THE UNIVERSITY OF NEBRASKA, :Lincoln, NE (US)

(21) Appl. No.: 13/982,149

(22) PCT Filed: Jan. 27, 2012
(86) PCT No.: PCT/US12/23030

§ 371 Date: Oct. 15, 2013

Related U.S. Application Data
(60) Provisional application No. 61/437,398, filed on Jan. 28, 2011.

(51) Int. Cl. A61K 38/13; A61K 31/713; A61K 45/06; A61K 39/395

(52) U.S. Cl. CPC A61K 38/13; A61K 39/3955; A61K 31/713; A61K 45/06
USPC 424/134.1; 530/389.2; 536/24.5; 530/317; 424/158.1; 514/44 A; 514/20.5; 424/133.1

(57) ABSTRACT

Methods and compositions for modulating cyclophilin D, e.g., at least one cyclophilin D biological activity, are provided. Modulation of cyclophilin D is useful in preventing or treating obesity, an overweight condition, or in accommodating a desire to lose weight as well as being useful in treating a variety of kidney diseases.

This application claims the benefit of Provisional U.S. Patent Application No. 61/437,398, filed Jan. 28, 2011, which is incorporated herein by reference in its entirety.

This applicant contains, as a separate part of the disclosure, a Sequence Listing in computer-readable form (filename: 46425_SeqListing.txt, created Jan. 27, 2012; ASCII text file) which is incorporated by reference in its entirety.

### FIELD OF THE INVENTION

The disclosure relates to methods and compositions for inhibition of cyclophilin D (CypD).

### BACKGROUND OF THE INVENTION

The cyclophilins are a family of proteins that bind to Cyclosporine (Cyclosporin), an immunosuppressant frequently used to suppress transplant rejection after internal organ transplantation. CypA, CypB and CypC are cytosolic isozymes that isomerize peptide bonds involving prolyl residues. CypD is located in the mitochondrial matrix and is a component of the mitochondrial permeability transition pore. CypD binds to Cyclosporine A (CsA), and CsA has been reported to inhibit the opening of the pore, suggesting that CypD functions as a regulator of pore opening.

Given the potential significance of CypD to mitochondrial physiology, investigations of CypD have been undertaken and have led to proposals to inhibit CypD in various therapies to treat, e.g., both heart-ischemia reperfusion and adult brain ischemia-reperfusion injuries (Baines et al., Nature 434 (2005) 658-662; Nakagawa et al., Nature 434 (2005) 652-658). Additionally, CypD inhibition has been proposed as a therapeutic for autoimmune encephalitis (Forte et al., Proc. Natl. Acad. Sci. (USA) 104 (2007) 7558-7563). Inhibition of CypD may also protect against mitochondrial and neuronal perturbations observed in Alzheimer's disease patients (Forte, Proc. Natl. Acad. Sci. (USA) 104 (2007) 7558-7563). Other possibilities include the inhibition of CypD to treat amyotrophic lateral sclerosis (Lou Gehrig's disease; Martin et al., Exp. Neurol. 218 (2009) 333-346) and to treat muscular dystrophy linked to sacroglycan (Millay et al., Nat. Med. 14 (2008) 442-447), laminin (Id.), or collagen VI (Palma et al., Hum. Mol. Genet. 18 (2009) 2024-2031) deficiency.

Obesity has become a leading preventable cause of disease and death in the United States, second only to tobacco use. Direct medical costs related to being overweight and obese have exceeded $100 billion per year. The rising medical costs associated with comorbidities, and chronic diseases secondary to obesity could overwhelm the financial stability of Medicare by 2020. Despite the urgent need for an effective treatment, no effective pharmacological therapies are currently available to prevent or treat obesity.

Currently, only two anti-obesity medications, orlistat and sibutramine, are approved by the FDA for long term use. Clinical trials using these drugs demonstrated modest weight loss, although none of the trials achieved the NIH effectiveness threshold. The use of sibutramine is now banned in Europe and is cautioned by the U.S. FDA as a result of increased cardiovascular risk associated with its use. The failure to develop effective pharmacological interventions to date is mainly due to the lack of a good understanding of the basic mechanisms of metabolic energy balance. Consequently, the relevant molecular targets for effective intervention remain elusive.

Acute kidney injury (AKI) is a devastating clinical syndrome with a relatively high mortality rate. The incidence of AKI among patients admitted to intensive care units is approximately 30% and is frequently associated with high mortality rates (50 to 80%). Approximately 50% of all cases of AKI result from renal ischemia-reperfusion injury. Despite the urgent need for an effective treatment, no clinically approved methods are available to treat or reverse the disease at the present time, mainly because the pathophysiology underlying the development of renal ischemia-reperfusion injury in AKI is still incompletely understood.

Many rational pharmacological interventions based on pathogenic factors that induce endothelial and epithelial cell injury, such as vasoconstriction, vascular congestion, leukostasis, and reactive oxygen species generation have failed or are inconclusive.

Renal dysfunction or kidney disease, e.g., nephrotoxicity, can also arise as a side effect of the therapeutic treatment of disease, for example the treatment of cancer. One of the most commonly used drugs for the treatment of malignant tumors is cisplatin (cisplatinum), which is in widespread use in the treatment of malignant tumors in testis, ovary, bladder, head and neck, breast and many other tissues/organs. Although effective, the use of cisplatin is limited by its severe side effects in normal tissues. Among these side effects the major side effect during cisplatin treatment is nephrotoxicity. After cisplatin treatment, approximately one-third of patients develop renal dysfunction, resulting in acute renal failure.

Both apoptotic and necrotic cell death play key roles in cisplatin nephrotoxicity and are simultaneously induced in kidney tubules after cisplatin injection. The mode of cell death is dependent mainly on cisplatin concentration. Most recent studies have focused on the mechanisms of apoptosis induced by cisplatin in kidney tubular cells, and much less on necrotic pathways.

Fibrotic diseases (e.g., chronic kidney disease) account for up to 45% of deaths in the developed world, yet there are no approved anti-fibrotic therapies. Current treatments for fibrotic diseases are relatively ineffective because they target multiple pathways leading to an inflammatory response, and these pathways are believed to be distinct from those pathways driving fibrogenesis. Identification of the primary signals or the core pathway that are essential to convert an initial stimulus to the development of fibrosis, and their targeting may be required to limit progression.

Currently, there are no treatments available for preventing or reversing renal fibrosis. Approximately 10% of the general population is affected by progressive kidney disease which leads to chronic renal failure and, eventually, end-stage renal disease. Treatment of progressive kidney disease can reduce the morbidity and mortality of people afflicted with chronic kidney disease.

Thus, a need exists to develop new methods and compositions for treating and preventing obesity. Additionally, a need exists to develop new methods and compositions for treating and preventing cisplatinum-induced renal toxicity. A need also exists to develop new methods and compositions for treating and preventing renal fibrosis. In general, a need continues to exist for therapeutics and both prophylactic and treatment methods for kidney injury or disease.

### SUMMARY OF THE INVENTION

The disclosure satisfies at least one of the aforementioned needs in the art by providing methods and compositions for the treatment and/or prevention of obesity and various kidney disorders, including kidney diseases. Compositions according to the disclosure modulate the activity of CypD, including modulation of CypD expression. (Cyclophilin D or CypD is also known as Peptidylprolyl Isomerase D or PPID, CYP-40 and Rotamase D (EC 5.2.1.8).) Methods according to the disclosure comprise administration of an effective amount of a modulator of CypD, with that effective amount being an amount sufficient to prevent and/or treat a disorder according to the disclosure. The compositions and methods provided by the disclosure will alleviate the pain and suffering of afflicted individuals, thereby improving their quality of life while alleviating the financial, psychological and physical burdens imposed on modern healthcare systems.

One aspect of the disclosure provides a modulator of cyclophilin D biological activity for use in the treatment of a subject having a disorder selected from the group consisting of obesity and kidney disease. In some embodiments the disorder is obesity, such as high-fat-diet-induced obesity.

In a related aspect, the disclosure provides a modulator for use in a method of stimulating weight loss in a subject comprising administering an effective amount of a composition comprising a modulator of cyclophilin D biological activity, such as a modulator of cyclophilin D protein activity. Another aspect is a modulator for use in a method of reducing the weight of an overweight subject comprising administering an effective amount of a composition comprising a modulator of cyclophilin D biological activity, such as a modulator of cyclophilin D protein activity. In still another aspect, the disclosure provides a modulator useful in a method for treating or preventing obesity in a subject comprising administering an effective amount of a composition comprising a modulator of cyclophilin D biological activity, e.g., a modulator of cyclophilin D protein activity.

Related aspects according to the disclosure provide a modulator for use in a method of increasing energy expenditure in a subject comprising administering a therapeutically effective amount of a modulator of cyclophilin D activity, such as a modulator of cyclophilin D protein activity. Also provided is a modulator for use in a method of increasing basal metabolic rate in a subject comprising administering a therapeutically effective amount of a modulator of cyclophilin D activity, e.g., a modulator of cyclophilin D protein activity. The disclosure further provides a modulator for use in a method of increasing heat production in a subject comprising administering a therapeutically effective amount of a modulator of cyclophilin D activity, such as a modulator of cyclophilin D protein activity. Additionally, the disclosure provides a modulator for use in a method of increasing total energy expenditure in a subject comprising administering a therapeutically effective amount of a modulator of cyclophilin D activity, e.g., a modulator of cyclophilin D protein activity. In a related aspect, the disclosure provides a modulator for use in a method of increasing the expenditure of fat energy in a subject comprising administering a therapeutically effective amount of a modulator of cyclophilin D activity, such as a modulator of cyclophilin D protein activity.

In some embodiments of the modulator of cyclophilin D biological activity, the modulator is useful in the treatment of a subject having a kidney disease. An exemplary kidney disease amenable to treatment (i.e., prophylaxis or therapy) is a kidney disease selected from the group consisting of renal fibrosis, nephrotoxicity, obstructive nephropathy, cisplatin-induced renal disease, acute renal failure, chronic renal failure, renal glomerulopathy and end-stage renal disease.

In embodiments according to this aspect of the disclosure, treatment is provided to a mammal, such as a human, or a mammalian pet, farm animal or zoo animal. In some embodiments, the modulator alters the expression level of a cyclophilin D coding region, thereby modulating cyclophilin D biological activity. In various embodiments, the modulator decreases cyclophilin D biological activity. Exemplary modulators of cypD expression include, but are not limited to, a modulator selected from the group consisting of siRNA (e.g., sc-44892 Cyclophilin D siRNA; Santa Cruz Biotechnology, Inc.), shRNA, miRNA, antisense nucleic acid and triplex-forming nucleic acid.

Suitable modulators for modulating CypD activity include, but are not limited to, a modulator selected from the group consisting of an antibody, a peptide, a non-toxic small molecule, cyclosporine A, a cyclosporine A derivative or analog, a non-immunosuppressive cyclosporine analog (NICAM), and a quinoxaline derivative. Nonlimiting examples of such suitable modulators include a modulator selected from the group consisting of 2,3-di(furan-2-yl)-6-ethoxycarbonylaminoquinoxaline, 2,3-di(furan-2-yl)-6-N—(N′,N′-diethylcarbamoyl)aminoquinoxaline, 2,3-di(furan-2-yl)-6-((R)-3-ethyoxycarbonyl-piperidino) carbonylaminoquinoxaline, 2,3-di(furan-2-yl)-6-((S)-3-ethyoxycarbonyl-piperidino) carbonylaminoquinoxaline, 2,3-di(furan-2-yl)-6-(pyrrolidin-1-yl) carbonylaminoquinoxaline, 2,3-di(furan-2-yl)-6-morpholinocarbonylaminoquinoxaline, 2,3-di(furan-2-yl)-6-N—(N′,N′-diisopropylcarbamoyl)aminoquinoxaline, Debio 025, N-methyl-4-isoleucine-cyclosporin (NIM811), N-methyl-4-valine-cyclosporin (PKF220-384), Antamanide, SCY-635, a polyketide, 3S-[3R*[E(1S*,3S*,4S*)],4S*,5R*,8S*,9E,12R*,14R*,15S*,16R*,18S*,19S*,26aR*-5,6,8,11,12,13,14,15,16,17,18,19,24,25,26,26a-hexadecahydro-5,19-dihydroxy-3-[2-(4-hydroxy-3-methoxycyclohexyl)-1-methylethenyl]-14,16-dimethoxy-4,10,12,18-tetramethyl-8-(2-propenyl)-15,19-epoxy-3H-pyrido[2,1-c][1,4]oxaazacyclotricosine-1,7,20,21(4H,23H)-tetrone monohydrate (FK-506, tacrolimus), L-685,818 (C18-OH, C21-ethyl-FK-506), NeuroSTAT® (an injectable formulation of cyclosporine A), CicloMulsion®, ascomycin (FK-520, L-683,590), [32-Arg] ascomycin, [U-13C] ascomycin, dihydro-FK-506 (FK-506D), C18-OH-ascomycin, A-119,435, U-13C-ascomycin, Leu-Ile,N-(glyoxyl)pipecolate ester, L-732,531, L-688,617, CaN A467-492 mitochondrial-targeted cyclosporine A (mtCsA), and Heat Shock Protein 60.

Another aspect of the disclosure is drawn to a modulator for use in a method of inhibiting collagen deposition comprising administering a therapeutically effective amount of an inhibitor of Cyclophilin D biological activity. A related aspect according to the disclosure is a modulator for inhibiting the expression of α-smooth muscle actin (α-SMA) comprising administering a therapeutically effective amount of an inhibitor of Cyclophilin D biological activity. Inhibition of collagen deposition and, independently, inhibition of α-SMA reduce the fibrogenesis associated with kidney disease and, thus, are useful in treating kidney fibrosis.

A modulator according to the disclosure includes a modulator formulated such that the modulator is in emulsified form, e.g., NeuroSTAT®.

In some embodiments wherein the modulator is administered to treat obesity, the modulator is in a composition further comprising a second obesity therapeutic selected from the group consisting of zonisamide, topiramate and rimonabant.

Additionally, in some embodiments wherein the modulator is administered to treat kidney disease, the modulator is in a composition further comprising a second kidney disease therapeutic selected from the group consisting of Minocycline, Rasagiline, Amifostine, IDN-6556 IDN-6734, VX-740, Adalimumab, Infliximab, Etanercept, ISIS 104828, INO-1001, nicotine amide, Edaravone, Idebenone, CEP-1347 and an apoptosis inhibitor.

Another aspect according to the disclosure is a method of preventing or treating a disorder in a subject wherein the disorder is selected from the group consisting of obesity and kidney disease comprising administering an effective amount of a composition comprising a modulator as disclosed above and defined herein. In some embodiments, the disorder is obesity or an overweight condition as assessed through objective medical criteria or through a subjective determination. In some embodiments of the method according to the disclosure, the obesity is high-fat-diet-induced obesity.

In some embodiments, the disclosure provides a method of stimulating weight loss in a subject comprising administering an effective amount of a composition comprising a modulator of cyclophilin D biological activity. The disclosure also contemplates a method of reducing the weight of an overweight subject comprising administering an effective amount of a composition comprising a modulator of cyclophilin D biological activity. Some embodiments are drawn to a method for treating or preventing obesity in a subject comprising administering an effective amount of a composition comprising a modulator of cyclophilin D biological activity.

Related aspects according to the disclosure provide a method of increasing energy expenditure in a subject comprising administering a therapeutically effective amount of a modulator of cyclophilin D activity, such as a modulator that modulates (e.g., inhibits) cyclophilin D translocation to the mitochondrial inner membrane. Also provided is a method of increasing basal metabolic rate in a subject comprising administering a therapeutically effective amount of a modulator of cyclophilin D activity. The disclosure further provides a method of increasing heat production in a subject comprising administering a therapeutically effective amount of a modulator of cyclophilin D activity. Additionally, the disclosure provides a method of increasing total energy expenditure in a subject comprising administering a therapeutically effective amount of a modulator of cyclophilin D activity. In a related aspect, the disclosure provides a method of increasing the expenditure of fat energy in a subject comprising administering a therapeutically effective amount of a modulator of cyclophilin D activity, such as a modulator of cyclophilin D expression.

In some embodiments of the disclosed method, the disorder is kidney disease, including but not limited to a kidney disease that is selected from the group consisting of renal fibrosis, nephrotoxicity, obstructive nephropathy, cisplatin-induced renal disease, acute renal failure, chronic renal failure, renal glomerulopathy and end-stage renal disease.

As in embodiments of the modulator aspect of the disclosure, embodiments of the method aspect include treatment that is provided to a mammal, such as a human, or a mammalian pet, farm animal or zoo animal. In some embodiments, the method comprises administration of a modulator that alters the expression level of a cyclophilin D coding region, thereby modulating cyclophilin D biological activity. In various embodiments, the method comprises administration of a modulator that decreases cyclophilin D biological activity. Exemplary modulators of cypD expression for use in the methods according to the disclosure include, but are not limited to, a modulator selected from the group consisting of siRNA (e.g., sc-44892 Cyclophilin D siRNA; Santa Cruz Biotechnology, Inc.), shRNA, miRNA, antisense nucleic acid and triplex-forming nucleic acid. The disclosed methods comprise administration of suitable modulators for modulating CypD activity, including, but not being limited to, a modulator selected from the group consisting of an antibody, a peptide, a non-toxic small molecule, cyclosporine A, a cyclosporine A derivative or analog, a NICAM and a quinoxaline derivative. Nonlimiting examples of such suitable modulators for use in the methods according to the disclosure include a modulator selected from the group consisting of 2,3-di(furan-2-yl)-6-ethoxycarbonylaminoquinoxaline, 2,3-di(furan-2-yl)-6-N—(N′,N′-diethylcarbamoyl)aminoquinoxaline, 2,3-di(furan-2-yl)-6-((R)-3-ethyoxycarbonyl-piperidino) carbonylaminoquinoxaline, 2,3-di(furan-2-yl)-6-((S)-3-ethyoxycarbonyl-piperidino) carbonylaminoquinoxaline, 2,3-di(furan-2-yl)-6-(pyrrolidin-1-yl) carbonylaminoquinoxaline, 2,3-di(furan-2-yl)-6-morpholinocarbonylaminoquinoxaline, 2,3-di(furan-2-yl)-6-N—(N′,N′-diisopropylcarbamoyl)aminoquinoxaline, Debio 025, N-methyl-4-isoleucine-cyclosporin (NIM811), N-methyl-4-valine-cyclosporin (PKF220-384), Antamanide, SCY-635, a polyketide, 3S-[3R*[E(1S*,3S*,4S*)],4S*,5R*,8S*,9E,12R*,14R*,15S*,16R*,18S*,19S*,26aR*-5,6,8,11,12,13,14,15,16,17,18,19,24,25,26,26a-hexadecahydro-5,19-dihydroxy-3,2-(4-hydroxy-3-methoxycyclohexyl)-1-methylethenyl]-14,16-dimethoxy-4,10,12,18-tetramethyl-8-(2-propenyl)-15,19-epoxy-3H-pyrido[2,1-c][1,4]oxaazacyclotricosine-1,7,20,21(4H,23H)-tetrone monohydrate (FK-506, tacrolimus), L-685,818 (C18-OH, C21-ethyl-FK-506), NeuroSTAT®, CicloMulsion®, ascomycin (FK-520, L-683,590), [32-Arg] ascomycin, [U-13C] ascomycin, dihydro-FK-506 (FK-506D), C18-OH-ascomycin, A-119,435, U-13C-ascomycin, Leu-Ile,N-(glyoxyl)pipecolate ester, L-732,531, L-688,617, CaN A467-492 mitochondrial-targeted cyclosporine A (mtCsA), and Heat Shock Protein 60.

Another aspect of the disclosure is drawn to a method of inhibiting collagen deposition comprising administering a therapeutically effective amount of an inhibitor of Cyclophilin D biological activity. A related aspect according to the disclosure is a method for inhibiting the expression of α-smooth muscle actin (α-SMA) comprising administering a therapeutically effective amount of an inhibitor of Cyclophilin D biological activity. As noted above, inhibition of collagen deposition and, independently, inhibition of α-SMA reduce the fibrogenesis associated with kidney disease and, thus, are useful in treating kidney fibrosis.

In some embodiments of the aspects of the disclosure drawn to methods (prevention or treatment), the method comprises administration of a therapeutically effective amount (sufficient to achieve a detectable prophylactic or treatment effect) of a modulator formulated such that the modulator is in emulsified form (e.g., NeuroSTAT®).

In some embodiments wherein the modulator is administered to treat obesity, the method comprises administration of an effective amount of a modulator that is in a composition further comprising a second obesity therapeutic selected from the group consisting of zonisamide, topiramate and rimonabant.

Additionally, in some embodiments of the method wherein the modulator is administered to prevent or treat kidney disease, the modulator is in a composition further comprising a second kidney disease therapeutic selected from the group consisting of Minocycline, Rasagiline, Amifostine, IDN-6556 IDN-6734, VX-740, Adalimumab, Infliximab, Etanercept, ISIS 104828, INO-1001, nicotine amide, Edaravone, Idebenone, CEP-1347 and an apoptosis inhibitor.

In some embodiments of the above-described method according to the disclosure, the modulator being administered indirectly modulates cyclophilin D activity by modulating the expression of cypD. Suitable modulators for use in these embodiments include the inhibitory nucleic acids disclosed below.

Yet another aspect of the disclosure is a composition comprising a modulator of cyclophilin D activity and a pharmaceutically acceptable carrier formulated for administration to treat or prevent a disorder selected from the group consisting of obesity and kidney disease. In some embodiments, the composition is useful for preventing or treating obesity.

In some embodiments, the composition is useful for the treatment or prevention of a kidney disorder, such as a composition useful for preventing or treating a disorder selected from the group consisting of renal fibrosis, nephrotoxicity, obstructive nephropathy, cisplatin-induced renal disease, acute renal failure, chronic renal failure, renal glomerulopathy and end-stage renal disease.

Compositions according to the disclosure include, but are not limited to, compositions comprising a modulator capable of modulating CypD expression selected from the group consisting of siRNA (e.g., sc-44892 Cyclophilin D siRNA; Santa Cruz Biotechnology, Inc.), shRNA, miRNA, antisense nucleic acid and triplex-forming nucleic acid. In some embodiments, the composition comprises a modulator capable of modulating CypD expression selected from the group consisting of an antibody, a peptide, a non-toxic small molecule, cyclosporine A, a cyclosporine A derivative or analog, a NICAM and a quinoxaline derivative. In exemplary embodiments, the composition comprises a modulator selected from the group consisting of 2,3-di(furan-2-yl)-6-ethoxycarbonylaminoquinoxaline, 2,3-di(furan-2-yl)-6-N—(N′,N′-diethylcarbamoyl)aminoquinoxaline, 2,3-di(furan-2-yl)-6-((R)-3-ethyoxycarbonyl-piperidino) carbonylaminoquinoxaline, 2,3-di(furan-2-yl)-6-((S)-3-ethyoxycarbonyl-piperidino) carbonylaminoquinoxaline, 2,3-di(furan-2-yl)-6-(pyrrolidin-1-yl)carbonylaminoquinoxaline, 2,3-di(furan-2-yl)-6-morpholinocarbonylaminoquinoxaline, 2,3-di(furan-2-yl)-6-N—(N′,N′-diisopropylcarbamoyl)aminoquinoxaline, Debio 025, N-methyl-4-isoleucine-cyclosporin (NIM811), N-methyl-4-valine-cyclosporin (PKF220-384), Antamanide, SCY-635, a polyketide, 3S-[3R*[E(1S*,3S*,4S*)],4S*,5R*,8S*,9E,12R*,14R*,15S*,16R*,18S*,19S*,26aR*-5,6,8,11,12,13,14,15,16,17,18,19,24,25,26,26a-hexadecahydro-5,19-dihydroxy-3-[2-(4-hydroxy-3-methoxycyclohexyl)-1-methylethenyl]-14,16-dimethoxy-4,10,12,18-tetramethyl-8-(2-propenyl)-15,19-epoxy-3H-pyrido[2,1-c][1,4]oxaazacyclotricosine-1,7,20,21(4H,23H)-tetrone monohydrate (FK-506, tacrolimus), L-685,818 (C18-OH, C21-ethyl-FK-506), NeuroSTAT®, CicloMulsion®, ascomycin (FK-520, L-683,590), [32-Arg] ascomycin, [U-13C] ascomycin, dihydro-FK-506 (FK-506D), C18-OH-ascomycin, A-119,435, U-13C-ascomycin, Leu-Ile,N-(glyoxyl)pipecolate ester, L-732,531, L-688,617, CaN A467-492 mitochondrial-targeted cyclosporine A (mtCsA), and Heat Shock Protein 60.

In some embodiments, the composition comprises a modulator that is in emulsified form, such as NeuroSTAT®.

Compositions according to the disclosure include compositions comprising modulators, wherein the modulator is in a composition further comprising a second obesity therapeutic selected from the group consisting of zonisamide, topiramate and rimonabant.

Compositions according to the disclosure also comprise a modulator that is in a composition further comprising a second kidney disease therapeutic selected from the group consisting of Minocycline, Rasagiline, Amifostine, IDN-6556 IDN-6734, VX-740, Adalimumab, Infliximab, Etanercept, ISIS 104828, INO-1001, nicotine amide, Edaravone, Idebenone, CEP-1347 and an apoptosis inhibitor.

Yet another aspect according to the disclosure is a kit for treating a disorder selected from the group consisting of obesity and kidney disease comprising a therapeutically effective amount of a modulator of cyclophilin D, a pharmaceutically acceptable carrier and instructions for use thereof in treating the disorder.

Other features and advantages of the present disclosure will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

FIG. 1 shows: A: Average body weights of CypD^{-/-} male mice compared to wild-type (WT) high-fat-diet-fed male mice. (* p<0.001; n=6-8). B: Average body weights of CypD^{-/-} female mice compared to WT high-fat-diet-fed female mice. * p<0.001 (n=6-8). C: The average body weight of WT and CypD^{-/-} male mice, and WT and CypD^{-/-} female mice, fed HF-diet for 19 weeks. Also shown are the percentages of fat mass (* p<0.01), lean mass (p>0.05), and water mass (* p<0.01) in WT and CypD^{-/-} male mice fed HF diet for 19 weeks as analyzed by the NMR method (n=4 per group).

FIG. 2 shows cumulative energy intakes. A: Cumulative energy intake adjusted for body mass in WT and CypD^{-/-} male mice on HF diet compared to WT mice on high-fat diet for 17 weeks (* p<0.05; n=8). B: Cumulative energy intake adjusted for body mass in WT and CypD^{-/-} female mice on HF diet compared to WT mice on high-fat diet for 17 weeks (* p<0.05; n=8).

FIG. 3 shows A: Comparison of the oxygen consumption for a period of 24 hours for WT and CypD^{-/-} mice that were fed HF diet for 19 weeks. Darkened bar area represents dark cycle. B: Comparison of the respiration quotient for a period of 24 hours for WT and CypD^{-/-} mice that were fed HF diet for 19 weeks.

FIG. 4 shows data relating to glucose and insulin metabolism. A: Comparison of glucose tolerance by IPGTT between WT and CypD-KO male mice at 19 weeks post-HF feeding, with blood glucose levels measured as a function of time. * p<0.05, n=4. B: Comparison of glucose tolerance between WT and CypD-KO male mice, with plasma insulin levels measured as a function of time. * p<0.05, n=4. C: Comparison of insulin tolerance by insulin tolerance test (ITT) between WT and CypD-KO male mice at 19 weeks post-HF feeding, with blood glucose levels measured as a function of time. * p<0.05, n=4. D: Comparison of insulin tolerance by insulin tolerance test (ITT) between WT and CypD-KO male mice at 19 weeks post-HF feeding, with plasma insulin levels measured as a function of time. * p<0.05, n=4.

FIG. 5: Effect of cyclosporine A (CsA) treatment in obesity development: Wild type (C57Bl/6) mice were divided into 5 groups and were fed either normal diet or high-fat diet. The mice were treated with blank, 5 mg/kg/day, or 20 mg/kg/day of the Neurovive® drug for 23 days. The drug as well as the blank was withdrawn at day 23 and the mice were allowed to continue on their respective diets for another 20 days. The effect of drug treatment and its withdrawal on the development of obesity is shown.

FIG. 6: Effect of CypD deficiency on fibrogenesis: CypD deficiency reduced collagen deposition as shown by Sirus-red staining (A and B) and by hydroxyl proline staining (C) in mouse kidneys during unilateral ureteral obstruction at 7 and 14 days. D and E: Western blot analysis of alpha-smooth muscle actin (a-SMA) expression during unilateral ureteral obstruction.

FIG. 7: Effect of CypD gene ablation on renal functions after cisplatinum injury. Top panel: Renal function was evaluated by measuring blood urea nitrogen (BUN) levels in the serum at 3 and 5 days post-cisplatinum injury. Bottom panel: Renal function as assessed by measuring serum creatinine levels at 3 and 5 days post-cisplatinum injury. Abbreviations: sh refers to sham-operated, D3 is day 3 post-cisplatinum injury, and D5 is day 5 post-cisplatinum injury.

### DETAILED DESCRIPTION

The results disclosed herein demonstrate that cyclophilin D gene ablation as well as pharmacological inhibition prevent diet-induced obesity in mice. These results support therapeutics and therapies targeting CypD in methods for preventing or treating mammalian, e.g., human, obesity, including the morbidity and mortality associated with obesity.

Consistent with the role of CypD in energy metabolism, data disclosed herein demonstrate a key role for cyclophilin D in renal dysfunction, ATP depletion, and the consequent injury and cell death in ischemic kidneys. Inhibition of Cyclophilin D using pharmacological approaches is expected to provide an effective approach to the treatment of kidney disorders and, in particular, to lead to a reduction in the mortality and morbidity associated with human acute kidney injury or AKI.

The experimental results disclosed herein also demonstrate that inhibition of CypD inhibits necrotic cell death and prevents renal dysfunction and injury. Inhibition of Cyclophilin D using pharmacological approaches is expected to provide an effective approach to the treatment of cisplatinum-related nephrotoxicity.

Experimental results disclosed herein additionally demonstrate that cypD gene ablation prevents fibrosis and inflammation after unilateral ureteral obstruction, which is the standard model for renal fibrosis. Consequently, inhibition of CypD in patients at risk of developing fibrotic tissue, such as kidney, liver or heart fibrosis, is expected to provide an effective treatment for the disease.

Unless otherwise defined herein, scientific and technical terms employed in the disclosure shall have the meanings that are commonly understood and used by one of ordinary skill in the art. Unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Thus, for example, the reference to a particular modulator of CypD is a reference to one such modulator or a plurality of such modulators, including equivalents thereof. Also, the terms “at least one” and “one or more” have the same meaning and include one, two, three or more. The following terms, unless otherwise indicated, shall be understood to have the following meanings when used in the context of the disclosure.

Examples provided herein, including those following “such as” and “e.g.,” are considered as illustrative only of various aspects of the disclosure and embodiments thereof, without being specifically limited thereto. Any suitable equivalents, alternatives, and modifications thereof (including materials, substances, constructions, compositions, formulations, means, methods, conditions, etc.) known and/or available to one skilled in the art may be used or carried out in place of, or in combination with, those disclosed herein, and are considered to fall within the scope of the disclosure.

As used in the disclosure, the term “treating” or “treatment” refers to an intervention performed with the intention of preventing the further development of or altering the pathology of a disease or infection. Accordingly, “treatment” refers to both therapeutic treatment and prophylactic or preventative measures. Of course, when “treatment” is used in conjunction with a form of the separate term “prophylaxis,” it is understood that “treatment” refers to the narrower meaning of altering the pathology of a disease, infection, or condition. “Preventing” refers to a preventative measure taken with a subject not previously exposed to or infected with a particular pathogen, or otherwise not having a condition or disease. A therapeutic agent may directly decrease the pathology of a disease or infection, or render the disease or infection more susceptible to treatment by another therapeutic agent(s) or, for example, the host's immune system. Treatment of patients suffering from clinical, biochemical, radiological or subjective symptoms of a disease or infection may include alleviating some or all of such symptoms or reducing the predisposition to the disease. Improvement after treatment may be manifested as a decrease or elimination of one or more of such symptoms.

As used herein, the phrase “effective amount” or “therapeutically effective amount” is meant to refer to an amount of a therapeutic or prophylactic CypD modulator that would be appropriate for an embodiment of the disclosure, that will elicit the desired therapeutic or prophylactic effect or response, including alleviating some or all of at least one of such symptoms of disease or infection or reducing the predisposition to the disease or infection, when administered in accordance with the desired treatment regimen.

As used herein, “concurrent” administration of two therapeutic agents does not require that the agents be administered at the same time or by the same route, as long as there is an overlap in the time period during which the agents are exerting their therapeutic effect. Simultaneous or sequential administration is contemplated, as is administration on different days or weeks. “Prior” administration refers to administering a CypD modulator at some time before administering a second therapeutic agent, irrespective of whether the two therapeutic agents are exerting a therapeutic effect together. Moreover, “following” administration refers to administering a CypD modulator at some time after administering a second therapeutic agent, irrespective of whether the two therapeutic agents are exerting a therapeutic effect together.

“Mammal” for purposes of treatment and prevention refers to any animal classified as a mammal, including humans, domestic and farm animals, and zoo, sports, or pet animals, such as dogs, horses, cats, cows, etc. In one embodiment, the mammal is human.

### Cyclophilins

Cyclophilin was first identified as the receptor for cyclosporin A (used interchangeably with cyclosporine A herein), a potent immunosuppressive drug that is still widely used to prevent immunological rejection of transplanted tissue. The effects of the cyclosporin A:cyclophilin interaction have been well-documented. Cyclosporin A binds with a dissociation constant in the range of 10⁻⁸ mol/L, a value representing a relatively high degree of attraction (Handschumacher et al., Science 226:544 (1984)). While the disclosure is not bound by any particular theory, it appears that the complex formed between CyP and cyclosporin A exerts effects on the organism and cells, which leads to immunosuppression. The complex interacts with the cellular enzyme calcineurin, a calmodulin-dependent phosphatase, and the interaction prevents T-cell activation by blocking RNA transcription of the T-cell growth factor interleukin 2 (IL-2) (Palacios, J. Immunol. 128:337 (1982)). Without IL-2 to cause T-cell proliferation, specific T-cell populations cannot mount a strong immune response, resulting in immunosuppression.

A number of types of mammalian cyclophilins have been identified and cloned, including cyclophilins A, B, C, D, and cyclophilin-40 (Snyder et al., Nat. Med. 1:32-37 (1995); Friedman et al., Proc. Natl. Acad. Sci. (USA) 90:6815-6819 (1993)). Cyclophilin A is a 19 kD protein that is abundantly expressed in a wide variety of cells. Like the other cyclophilins, cyclophilin A binds the immunosuppressive agent cyclosporin A and possesses peptidyl-prolyl cis-trans isomerase (PPIase) and protein folding or “chaperone” activities. PPIase activity catalyzes the conversion of proline residues in a protein from the cis to the trans conformation (Fischer, et al., Biomed. Biochem. Acta 43:1101-1112 (1984)). Cyclophilin B possesses an N-terminal signal sequence that directs translocation into the endoplasmic reticulum of the cell. The 23 kD cyclophilin C is found in the cytosol of the cell. Cyclophilin D, at 18 kD, appears to target its actions in the mitochondria. Cyclophilin-40 is a component of the inactivated form of a glucocorticoid receptor.

Immunophilins were discovered because of their interaction with known therapeutic drugs. Thus, knowledge about the interaction between drug and protein spawned a number of drug discovery efforts. Initially, the focus was on identifying new immunosuppressive drugs. A number of facts have influenced the search for improved immunosuppressive drugs. One factor was the importance of proline. The native substrate for the PPIase activity in cells is the amino acid proline in a protein. Cyclophilins A-D all contain a conserved proline binding site. The conversion between the cis and trans forms of proline, which PPIase performs, allows a protein to change shape and fold properly.

The first identified ligand for cyclophilins, however, was cyclosporin A, which is a cyclic peptide that does not contain a proline. Both FK-506 and rapamycin, which bind FKBP (FK-506 Binding Protein), are also cyclic antibiotics, but FK-506 and rapamycin are non-peptidic macrolide antibiotics. The FKBP proteins also possess PPIase activity, although the FKBPs share no significant sequence homology to cyclophilins (CyPs). Since FK-506 is a more potent immunosuppressive compound than cyclosporin A, a number of analogs of FK-506 have been developed.

Cyclophilin D (CypD) is also referred to as peptidylprolyl isomerase F (PPIF, cyclophilin F (mitochondrial form), Genbank Accession Nos. BC005020, M80254, AAA58434, AAH05020) and includes all forms thereof, including biologically active analogs, derivatives, fragments and variants. Cyclophilin D is found in the matrix and the inner membrane of mitochondria. Identification of cypD was prompted by the demonstration that CsA affects mitochondrial Ca²⁺ fluxes through an effect that could be traced to desensitization of the mitochondrial permeability transition pore (MPTP), an inner membrane high-conductance channel whose molecular composition remains an unanswered question. Genetic ablation of the Ppif gene (which encodes for CypD) in the mouse has demonstrated that CypD is the mitochondrial receptor for CsA, and that it is responsible for modulation of the MPTP, but it is not a structural pore component. The CypD-bound form of the MPTP displays a higher probability of opening and allowing molecules less than 1500 Da to enter non-selectively through the pore.

### CypD and Obesity

When dietary energy is maintained at a constant level, weight gain or loss depends on the energy expenditure through exercise and other obligatory bodily functions (e.g., thermoregulation) and on the coupling efficiency. Coupling efficiency is defined as the proportion of the calories burned and oxygen consumed that is coupled to ATP synthesis. One of the mechanisms by which metabolic efficiency can be lowered is to activate futile cycles of ATP synthesis and hydrolysis. Proton leaks have been reported to account for 26% of resting energy expenditure in isolated hepatocytes and up to 50% in perfused rat skeletal muscle [Rolfe, D. F. et al., Physiol. Rev., 77: 731-58 (1997)]. Although the mechanism for the proton leak is not defined, the adenine nucleotide translocase (ANT), involved in export of ATP from mitochondria, is implicated [Brand, M. D., et al., Biochem. J., 392: 353-62 (2005)]. In addition, oxidative phosphorylation is uncoupled by inducible proton leak in specialized tissues such as brown fat by uncoupling proteins (UCPs) to cause adaptive thermogenesis.

Obesity is associated with diminished brown fat activity [Trayhurn, P., et al., Pflugers Arch., 380: 227-32 (1979)] and it was proposed that a malfunction of the brown-fat-specific uncoupling by UCP-1 may explain this phenomenon. Interestingly, UCP-1 gene deletion was associated with lack of ability to respond to β-adrenergic stimulated thermogenesis during acclimation to cold [Golozoubova, V., et al., Am. J. Physiol. Endocrinol. Metab., 291: E350-7 (2006)], but not with an obese phenotype [Enerback, S., et al., Nature, 387: 90-4 (1997), Liu, X., et al., J. Clin. Invest., 111: 399-407 (2003)]. A recent report, however, demonstrated that at thermoneutrality, the metabolic efficiency was increased in UCP-1 deficient mice and they had an obesogenic phenotype [Feldmann, H. M., et al., Cell Metab., 9: 203-9 (2009)], rekindling a role for UCP1 in bioenergetics.

Another possible mechanism by which leakage of protons may occur is by the intermittent opening of the mitochondrial permeability transition pore (MPTP). MPTP opening allows small molecules below the size of 1500 Da to pass through the inner mitochondrial membrane, causing disruption of the transmembrane potential and proton gradient [Halestrap, A., Biochem. Soc. Symp., 66: 181-203 (1999)]. CypD is a critical component of the MPTP that can modulate the permeability of the channel in response to various stress stimuli [Halestrap, A. P., Biochem. Soc. Trans., 34: 232-7 (2006)].

A role for MPTP in diet-induced obesity has not been investigated. Modulation of MPTP by CypD could be an alternate mechanism by which energy metabolism is uncoupled and adaptive thermogenesis could occur. As disclosed herein, the possibility was tested by feeding a high fat diet to CypD-deficient mice and it was determined whether body weight, food intake, energy expenditure, or body fat stores were altered. Contrary to expectations, results demonstrated that CypD-deficient mice are resistant to obesity.

### CypD and Kidney Disorders

The term “disorder” refers to a genetic or environmental risk of or propensity for developing symptoms or abnormal clinical indicators. A disorder is any condition that would benefit from treatment with a CypD modulator according to the disclosure. This includes chronic and acute disorders or diseases including those pathological conditions which predispose the mammal to the disorder in question.

Various embodiments of the disclosure include treatment of kidney disorders that include, but are not limited to, renal fibrosis, nephrotoxicity, obstructive nephropathy, acute renal failure, chronic renal failure. In another embodiment, renal toxicity resulting from exposure to chemotherapeutic agents such as cisplatinum is contemplated as a disorder amenable to treatment.

Kidney fibrosis is a typical characteristic of chronic renal disease. The exact mechanisms by which progressive kidney disease occurs has not been well defined. The roles of mitochondria, MPTP and CypD have not been previously investigated. Studies disclosed herein demonstrate a protective effect of CypD gene ablation on fibrogenesis in the UUO model.

Cisplatinum, or cisplatin, nephrotoxicity can involve tubular epithelial cell toxicity, vasoconstriction in the renal microvasculature, and proinflammatory effects. A role for cypD in cisplatinum nephrotoxicity has not been described previously. Data disclosed herein demonstrate that genetic ablation of cypD prevented cisplatinum nephrotoxicity. It should be noted that Cyclosporin A (CsA), a potent inhibitor of the MPTP, has been shown to be protective in cisplatin nephrotoxicity in rats by Campbell et al., Toxicology; 114:11-7, 1996. Renal insufficiency induced by cisplatin, as assessed by serum creatinine and urea levels, was significantly alleviated in rats 4 days after i.p. injections of cisplatin (5 mg/kg body wt) and CsA (50 micrograms/kg body wt), compared to rats injected with cisplatin alone.

### Modulators of Cyclophilin D

### Modulators of Cyclophilin D Biological Activity

In various embodiments of the disclosure, CypD biological activity is modulated according to the methods described herein. Modulation of activity includes an observed increase or decrease of activity. In a preferred embodiment, CypD biological activity is decreased. Modulators of CypD biological activity include, but are not limited to, agents that bind to CypD, agents that prevent or reduce mitochondrial permeability transition pore (MPTP) opening, and agents that prevent or reduce the influx of cytosolic molecules into the mitochondrial matrix. In various embodiments, modulators include polypeptides such as antibodies, small peptides, or small molecules.

The term “antibody” is used in the broadest sense and includes fully assembled antibodies, monoclonal antibodies, polyclonal antibodies, multispecific antibodies (e.g., bispecific antibodies), antibody fragments that can bind antigen (e.g., Fab′, F′(ab)2, Fv, single chain antibodies, diabodies), camel bodies and recombinant peptides comprising the forgoing as long as they exhibit the desired biological activity. Antibody fragments may be produced using recombinant DNA techniques or by enzymatic or chemical cleavage of intact antibodies and are described further below. Nonlimiting examples of monoclonal antibodies include murine, chimeric, humanized, human, and Human Engineered™ immunoglobulins, antibodies, chimeric fusion proteins having sequences derived from immunoglobulins, or muteins or derivatives thereof, each described further below. Multimers or aggregates of intact molecules and/or fragments, including chemically derivatized antibodies, are contemplated. Antibodies of any isotype class or subclass are contemplated according to the disclosure.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations that typically include different antibodies directed against different determinants (epitopes), each monoclonal antibody is directed against a single determinant on the antigen. In addition to their specificity, the monoclonal antibodies are advantageous in that they are synthesized in a homogeneous culture, uncontaminated by other immunoglobulins with different specificities and characteristics.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the disclosure may be made by the hybridoma method first described by Kohler et al., Nature, 256:495 (1975), or may be made by recombinant DNA methods (see, e.g., U.S. Pat. No. 4,816,567, incorporated herein by reference). The “monoclonal antibodies” may also be recombinant, chimeric, humanized, human, Human Engineered™, or antibody fragments, for example.

Commercially available CypD antibodies, e.g., Santa Cruz and Mitoscience anti-CypD antibodies, are contemplated according to the disclosure for use in the methods described herein. Such antibodies may, for example, be humanized according to known techniques and modified and/or formulated to allow delivery and intracellular contact with CypD.

Small peptides or oligopeptides are also contemplated by the disclosure. The term “oligopeptide” refers to a peptide that is at least about 5 amino acids in length; for example at least 10 amino acids in length; for example at least about 20 amino acids in length; and at least about 50 amino acids in length. In various embodiments of the disclosure, the oligopeptide is cyclic (e.g., a ring system containing multiple amino acids and/or amino acid homologs and derivatives and/or intramolecular cyclizations) or acyclic (e.g., linear insofar as the N- and C-termini are not linked by, for example, a peptide bond, nor the presence of intermolecular cyclizations).

Cyclosporine A (CsA) binds to CypD to block the opening of MPTP. CsA is an immunosuppressant drug widely used in post-allogeneic organ transplant to reduce the activity of the immune system, and therefore the risk of organ rejection. It is a cyclic nonribosomal peptide of 11 amino acids and contains a single D-amino acid, which are rarely encountered in nature. Numerous CsA analogs have been reported in the literature, and each such analog is contemplated as suitable for use as a prophylactic or therapeutic in the methods according to the disclosure. As used herein, an “analog” or “CsA analog” has the same functional and/or biological properties of CsA, such as binding properties, and/or the same structural basis, such as a cyclic peptidic backbone, or a basic polymeric unit. In this way, “analog” refers to a compound having a chemically modified form of a principal compound or class thereof, which maintains significant structure and the pharmaceutical and/or pharmacological activities characteristic of the principal compound or class.

A “derivative,” for example, is a type of structural analog, also known as chemical analog or simply analog, which is a chemical compound having a structure similar to that of another one, but differing from it in respect of a certain component. It can differ in one or more atoms, functional groups, or substructures, which are replaced with other atoms, groups, or substructures. A structural analog can be viewed as being formed, at least theoretically, from the parent, or reference, compound by being modified, e.g., chemically.

In another embodiment of the disclosure, Alisporivir (e.g., Debio 025, a cyclophilin inhibitor) is contemplated. Its structure is reminiscent of, and it is synthesized from, CsA. (Tiepolo, T., et al., Brit. J. Pharmacol., 157:1045-1052 (2009); incorporated by reference in its entirety.) In another embodiment, NIM811, which is structurally very similar to CsA, with an isobutyl group replaced by a sec-butyl group at position 4, is contemplated. (Ma, S., et al., Antimicrob. Agents Chemother., 50(9):2976-2982 (2006); incorporated by reference in its entirety.)

In various other embodiments of the methods according to the disclosure, CsA analogs such as those described in any of WO 2011/082289, U.S. Pat. Pub. No. 2011/0144005, WO 2010/002428, U.S. Pat. No. 6,809,077, and U.S. Pat. Pub. No. 2011/0008286 each hereby incorporated by reference in its entirety, is contemplated.

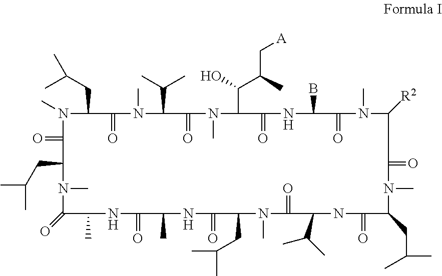

Exemplary compounds useful in these embodiments include compounds of Formula I as provided above wherein A represents (E)-CH═CHR or —CH₂CH₂R, wherein R represents methyl, —CH₂SH, —CH₂(thioalkyl), carboxyl or alkoxycarbonyl; B represents methyl, ethyl, 1-hydroxyethyl, isopropyl or n-propyl; R₁ represents: methyl substituted by R₂₁; straight- or branched-chain alkyl containing from two to six carbon atom substituted by one or more groups R₂₂ which may be the same or different; straight- or branched-chain alkenyl containing from four to eight carbon atoms, or straight- or branched-chain alkenyl containing from three to eight carbon atoms substituted by one or more groups R₂₃ which may be the same or different; straight- or branched-chain alkynyl containing from three to six carbon atoms optionally substituted by one or more groups which may be the same or different selected from the group consisting of halogen, hydroxyl, amino, N-monoalkylamino and N,N-dialkylamino; cycloalkyl containing from three to six carbon atoms optionally substituted by one or more groups which may be the same or different selected from the group consisting of halogen, hydroxyl, amino, N-monoalkylamino and N,N-dialkylamino; or straight- or branched-chain alkoxycarbonyl containing from two to six carbon atoms; R2 represents: straight- or branched-chain alkyl containing from one to six carbon atoms; straight- or branched-chain alkenyl containing from three to six carbon atoms; or straight- or branched-chain alkynyl containing from two to six carbon atoms; R₂₁ represents halogen; hydroxyl; alkoxycarbonyl; —C(═O)NR₃R₄; —OR₅; formyl; —C(═O)R₅; —S(O)ₙR₅; —NR₃R₄; or cycloalkyl containing from three to six carbon atoms optionally substituted by one or more groups which may be the same or different selected from the group consisting of halogen, hydroxyl, amino, N-monoalkylamino and N,N-dialkylamino; or R₂₁ represents a carbon-linked saturated or unsaturated heterocyclic ring containing from four to six ring atoms, which ring contains from one to three heteroatoms which may be the same or different selected from the group consisting of nitrogen, oxygen and sulfur, which ring may be optionally substituted by from one to four groups which may be the same or different selected from the group consisting of alkyl, halogen, alkoxy, amino, carboxyl and alkyl, which alkyl is substituted by amino, N-alkylamino or N,N-dialkylamino; R₂₂ and R₂₃, which may be the same or different, each represents halogen; hydroxyl; —OR₅; carboxyl; alkoxycarbonyl; —C(═O)NR₃R₄; formyl; —C(═O)R₅; —S(O)ₙR₅; —NR₃R₄; —NR₆(CH₂)ₘNR₃R₄; benzyl optionally substituted by from one to five groups which may be the same or different selected from the group consisting of alkyl, haloalkyl, halogen, hydroxyl, alkoxy, amino, N-alkylamino, N,N-dialkylamino, carboxyl and alkoxycarbonyl; or cycloalkyl containing from three to six carbon atoms optionally substituted by one or more groups which may be the same or different selected from the group consisting of halogen, hydroxyl, amino, N-monoalkylamino and N,N-dialkylamino; R₃ and R₄, which may be the same or different, each represent: hydrogen; —C(═O)R₅; —S(O)₂R₅; straight- or branched-chain alkyl containing from one to six carbon atoms; straight- or branched-chain alkenyl or alkynyl containing from two to four carbon atoms; or cycloalkyl containing from three to six carbon atoms optionally substituted by straight- or branched-chain alkyl containing from one to six carbon atoms; or R₃ and R₄, together with the nitrogen atom to which they are attached, form a saturated heterocyclic ring containing from four to six ring atoms, which ring may optionally contain another heteroatom selected from the group consisting of nitrogen, oxygen and sulfur, which ring may be optionally substituted by from one to four groups which may be the same or different selected from the group consisting of alkyl, phenyl and benzyl; R₅ represents: straight- or branched-chain alkyl containing from one to six carbon atoms; aryl optionally substituted by from one to five groups which may be the same or different selected from the group consisting of alkyl, haloalkyl, halogen, hydroxyl, alkoxy, amino, N-alkylamino and N,N-dialkylamino; heteroaryl optionally substituted by from one to five groups which may be the same or different selected from the group consisting of alkyl, haloalkyl, halogen, hydroxyl, alkoxy, amino, N-alkylamino and N,N-dialkylamino; aralkyl, wherein the aryl ring is optionally substituted by from one to five groups which may be the same or different selected from the group consisting of halogen, amino, N-alkylamino, N,N-dialkylamino, alkoxy and haloalkyl, wherein the alkylene group attached to the aryl ring contains one to three carbon atoms; or heteroarylalkyl wherein the heteroaryl ring is optionally substituted by halogen, amino, N-alkylamino, N,N-dialkylamino, alkoxy or haloalkyl, wherein the alkylene group attached to the aryl ring contains one to three carbon atoms; R₆ represents hydrogen, straight- or branched-chain alkyl containing from one to six carbon atoms, cyano or alkylsulfonyl; m is an integer from one to four; and n is 0, 1 or 2 and pharmaceutically acceptable salts and solvates thereof.

Various embodiments of the modulator of CypD biological activity according to the disclosure are drawn to cyclosporin (used interchangeably with cyclosporine herein) analogs. Cyclosporin compounds (cyclosporin compounds and analogs thereof) constitute a chemical class that is now substantial and includes, for example, the naturally occurring cyclosporins A through Z, for example, [Thr]², [Val]², [Nva]² and [Nva]²-, [Nva]⁵-cyclosporin (also known as cyclosporins C, D, G and M respectively), [(D)MeVal]¹¹-cyclosporin (also known as cyclosporin H); as well as various non-natural cyclosporin derivatives and artificial or synthetic cyclosporin derivatives and artificial or synthetic cyclosporins including dihydrocyclosporins, in which the MeBmt-residue is saturated by hydrogenation; derivatized cyclosporins (e.g., cyclosporins in which the 3′-O-atom of the MeBmt-residue is acylated or a further substituent is introduced at the α-carbon atom of the sarcosyl residue at the 3-position); and cyclosporins in which variant amino acids are incorporated at specific positions within the peptide sequence, for example, [3-O-acetyl-MeBmt]¹-Cyclosporin (also known as Dihydro-cyclosporin D), [(D)Ser]⁸-Cyclosporin, [Melle]¹¹-Cyclosporin, [MeAla]⁶-Cyclosporin, and [(D) Pro]³-Cyclosporin.

Small molecules are also contemplated. For example, non-peptidic cyclophilin-binding compounds are described in U.S. Pat. Pub. No. 2003/0232815, incorporated by reference in its entirety. In particular, small molecules according to the disclosure include, but are not limited to, chemicals of Formula II and Formula III.

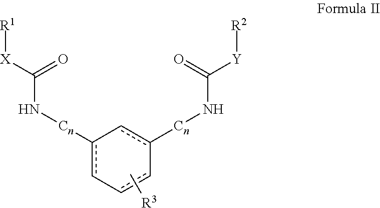

where n in Cₙ is 0 or 1; the dashed bond symbol represents an optional bond; X and Y may independently be N, NH, O, S, or a direct bond; R₁ is the same or different from R₂, and either can be one or more C1-C6 branched or straight chain alkyl or alkenyl groups; one or more C1-C3 branched or straight chain alkyl groups substituted by one or more Q groups; or one or more Q groups, where Q, which is optionally saturated, partially saturated, or aromatic, is a mono-, bi-, or tri-cyclic, carbo- or hetero-cyclic ring, wherein each ring may be optionally substituted in one to three positions with halo, hydroxyl, nitro, trifluoromethyl, acetyl, aminocarbonyl, methylsulfonyl, oxo, cyano, carboxy, C1-C6 straight or branched chain alkyl or alkenyl, C1-C4 alkoxy, C1-C4 alkenyloxy, phenoxy, benzyloxy, amino, or a combination thereof, and wherein the individual ring sizes are 5-6 atoms, and wherein each heterocyclic ring contains 1-6 heteroatoms selected from the group consisting of O, N, S, or a combination thereof; and R₃ may be one to three substituents chosen from the group consisting of halo, hydroxyl, nitro, trifluoromethyl, C1-C6 straight or branched chain alkyl or alkenyl, C1-C4 alkoxy, C1-C4 alkenyloxy, phenoxy, benzyloxy, amino, Q as defined above, or a combination thereof.

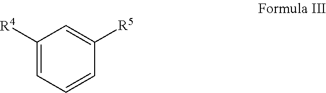

where R₄ and R₅ may independently be —N—SO₂—R, —SO₂—NRR, —O—R, —CO—N—R, —N—CO—R, —CO—R, wherein each R may independently be hydrogen, Q, or a C1-C6 branched or straight alkyl or alkenyl chain, which may be substituted in one or more positions by C3-C8 cycloalkyl or cycloalkenyl, hydroxyl, or carbonyl oxygen, and wherein the alkyl or alkenyl chain contains one or more carbon atoms that are either optionally substituted with Q, or optionally replaced by O, S, SO, SO₂, N, or NH; where Q, which is optionally saturated, partially saturated, or aromatic, is a mono-, bi-, or tri-cyclic, carbo- or hetero-cyclic ring, wherein each ring may be optionally substituted in one to five positions with halo, hydroxyl, nitro, trifluoromethyl, acetyl, aminocarbonyl, methylsulfonyl, oxo, cyano, carboxy, C1-C6 straight or branched chain alkyl or alkenyl, C1-C4 alkoxy, C1-C4 alkenyloxy, phenoxy, benzyloxy, amino, or a combination thereof, and wherein the individual ring sizes are 5-6 atoms, and wherein each heterocyclic ring contains 1-6 heteroatoms selected from the group consisting of O, N, S, or a combination thereof.

In various embodiments of the disclosure, polyketide cyclophilin inhibitors, such as those belonging to the sanglifehrin family, are contemplated. (Gregory, M. A., et al., Antimicrob. Agents Chemother. 55(5):1975-81 (2011); WO 2010/004304; each hereby incorporated by reference in its entirety.) Sanglifehrins are a group of naturally occurring cyclophilin binding polyketides that are structurally distinct from the cyclosporines and are produced by a microorganism amenable to biosynthetic engineering for lead optimization and large-scale production by fermentation. Exemplary sanglifehrens contemplated by the disclosure include FK506 (Tacrolimus), FK520, FK523, FK525, Pimecrolimus, Antascomycin, Tsukubamycin. Analogs of FK506 and FK520 include compounds having a 22-member ring structure with one lactone and one amide bond, with the N of the amide bond forming a 2-carboxylpiperidine or a 2-carboxylpyrrolidine. Also contemplated is a member of the sangamides, with the sangamides being derivatives of sanglifehren A available from Biotica. Further contemplated is BC556, also available from Biotica.

CypD inhibitors derived from quinoxaline are also contemplated by the disclosure. (Guo, H., et al., Acta Pharmacologica Sinica, 26(1):1201-1211 (2005); incorporated by reference in its entirety.) Exemplary CypD inhibitors derived from quinoxaline include 2,3-di(furan-2-yl)-6-ethoxycarbonylaminoquinoxaline, 2,3-di(furan-2-yl)-6-N—(N′,N′-diethylcarbamoyl)aminoquinoxaline, 2,3-di(furan-2-yl)-6-((R)-3-ethyoxycarbonyl-piperidino) carbonylaminoquinoxaline, 2,3-di(furan-2-yl)-6-((S)-3-ethyoxycarbonyl-piperidino) carbonylaminoquinoxaline, 2,3-di(furan-2-yl)-6-(pyrrolidin-1-yl) carbonylaminoquinoxaline, and 2,3-di(furan-2-yl)-6-morpholinocarbonylaminoquinoxaline, 2,3-di(furan-2-yl)-6-N—(N′,N′-diisopropylcarbamoyl)aminoquinoxaline.

### Modulators of Cyclophilin D Expression

In various embodiments of the disclosure, expression of CypD is modulated according to the methods described herein. Modulation of expression includes an observed increase or decrease in expression. In a preferred embodiment, the expression of CypD is decreased by a modulator. Modulators of CypD expression include, but are not limited to, agents that interfere with CypD gene transcription, CypD mRNA translation or CypD protein production. In various embodiments, modulators include antisense nucleic acids (DNA or RNA), small interfering RNAs (siRNA), ribozymes, deoxyribozymes, microRNA, locked nucleic acids (LNA), peptide-nucleic acids (PNA), aptamers, peptides (including proteins, e.g., antibodies), and small molecules.

### Compositions and Methods of Administering

In various embodiments of the disclosure, a composition comprising one or more “therapeutic agents” or “active agents,” e.g., a CypD modulator, is provided.

In some embodiments, the active agent or therapeutic agent in the composition is CsA or a CsA analog as described herein. CsA exhibits very poor solubility in water and, as a consequence, suspension and emulsion forms of the drug have been developed for oral administration and for injection. For example, commercially available formulations of CsA that are contemplated for use in the methods described herein include, but are not limited to, Sandimmune (gelatin capsules, oral solution, or formulation for intravenous administration), Neoral (solution and gelatin capsules), Cicloral, Gengraf, and Deximune. In a preferred embodiment, NeuroSTAT® (CsA and a carrier medium which is free from cremophor and alcohol) is contemplated for use in the methods described herein and according to the disclosure.

“Prodrug” refers to any covalently bonded compound comprising the drug (e.g., a carrier) that is capable of releasing an active agent in vivo when administered to a subject. Prodrugs are known to enhance numerous desirable qualities (e.g., solubility, bioavailability, manufacturing, stability) of the active agents. Prodrugs may be prepared by modifying functional groups (e.g., hydroxy, amino, carboxyl, and/or sulfhydryl groups) present in the active agent in such a way that the modifications are reversible (e.g., modifier group cleaved), either in routine manipulation or in vivo, to provide the original active agent. The transformation in vivo may be, for example, the result of some metabolic process, such as chemical or enzymatic hydrolysis of a carboxylic, phosphoric or sulfate ester, or reduction or oxidation of a susceptible functionality.

In various embodiments, the composition comprises a pharmaceutically acceptable carrier, e.g., one or more solvents, dispersion media, coatings, antibacterial and antifungal agents, isotonic and absorption delaying agents, and the like, compatible with administration to a mammal, such as a human. Any carrier compatible with the excipient(s) and therapeutic agent(s) (e.g., CypD modulator) is suitable for use. Supplementary active compounds may also be incorporated into the compositions. A composition of the disclosure is formulated to be compatible with its intended route of administration. Examples of routes of administration include oral administration (ingestion) and parenteral administration, e.g., intravenous, intradermal, and intraperitoneal administration. Solutions or suspensions used for parenteral application may include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH may be adjusted with acids or bases, such as hydrochloric acid or sodium hydroxide.

Oral compositions generally include an inert diluent or an edible carrier. Oral formulations generally take the form of a pill, tablet, capsule (e.g., softgel capsule, solid-filled capsule, or liquid-filled capsule), solid lozenge, liquid-filled lozenge, mouth and/or throat drops or spray, effervescent tablets, orally disintegrating tablet, suspension, emulsion, syrup, elixir, or tincture. The composition may be contained in enteric forms to survive the stomach or further coated or mixed to be released in a particular region of the gastrointestinal tract by known methods. Solid oral dosage forms are typically swallowed immediately, or slowly dissolved in the mouth. Oral compositions may also be prepared using a fluid carrier for use as a mouthwash, wherein the compound in the fluid carrier is applied orally and swished and expectorated or swallowed. Oral formulations optionally contain any of the following ingredients, or compounds of a similar nature: a binder such as microcrystalline cellulose, gum tragacanth or gelatin; starch or lactose; a disintegrating agent such as alginic acid, Primogel™, or corn starch; a lubricant such as magnesium stearate; a glidant such as colloidal silicon dioxide; and/or a sweetening agent such as sucrose or saccharin.

Optionally, the composition is formulated as a “liquid respiratory composition,” i.e., a composition in a form that is deliverable to a mammal via the oral cavity, mouth, throat, nasal passage or combinations thereof. These compositions can be delivered by a delivery device selected from droppers, pump, sprayers, liquid dropper, spoon, cup, squeezable sachets, power shots, and other packaging and equipment, and combinations thereof. In some embodiments, the liquid respiratory composition comprises the therapeutic agent, excipient, a thickening polymer (e.g., xanthan gum, cellulosic polymers such as carboxymethycellulose (CMC), hydroxethylcellulose, hydroxymethylcellulose, and hydroxypropylmethylcellulose, carrageenan, polyacrylic acid, cross-linked polyacrylic acid such as Carbopol®, polycarbophil, alginate, clay, and combinations thereof), and optionally a mucoadhesive polymer (e.g., polyvinylpyrrolidone (Povidone), methyl vinyl ether copolymer of maleic anhydride (Gantrez®), guar gum, gum tragacanth, polydextrose, cationic polymers, poly(ethylene oxide), poly(ethylene glycol), poly(vinyl alcohol), poly(acrylic acid), cross-linked polyacrylic acid such as Carbopol®, polycarbophil, poly(hydroxylethyl methacrylate), chitosan, cellulosic polymers such as carboxymethycellulose (CMC), hydroxethylcellulose, hydroxymethylcellulose, and hydroxypropylmethylcellulose, and combinations thereof). The composition is preferably a non-Newtonian liquid that exhibits zero shear viscosity from about 100 centiPoise (cP) to about 1,000,000 cP, from about 100 cP to about 500,000 cP, from about 100 cP to about 100,000 cP, from about 100 cP to about 50,000 cP, from about 200 cP to about 20,000 cP, from about 1,000 to about 10,000 cP at a temperature of about 37° C., as measured according to the Shear Viscosity Method. The pH range of the formulation is generally from about 1 to about 8.5, from about 1 to about 7, from about 2 to about 6.5, and from about 4 to about 6.

Pharmaceutical compositions suitable for injectable use include sterile aqueous solutions (where the therapeutic agent or CypD modulator is water-soluble), or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate-buffered saline (PBS). In all cases, the composition is sterile and fluid to allow syringability. The carrier may be a solvent or dispersion medium containing, for example, water, ethanol, polyol (for example, glycerol, propylene glycol, and liquid polyethylene glycol, and the like), and suitable mixtures thereof. Fluidity is maintained, for example, by the use of a coating such as lecithin, by the maintenance of the required particle size in the case of dispersion, and by the use of surfactants. Prevention of the action of microorganisms may be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions may be brought about by including in the composition an agent that delays absorption, for example, aluminum monostearate and gelatin. The injectable preparations may be enclosed in ampules, disposable syringes or multiple dose vials made of glass or plastic.

In one embodiment, the components of the composition are prepared with carriers that will protect the components against rapid elimination from the body, such as a controlled-release formulation, including implants and microencapsulated delivery systems. Biodegradable, biocompatible polymers may be used, such as ethylene vinyl acetate, polyanhydrides, polyglycolic acid, collagen, polyorthoesters, and polylactic acid.

The formulation is provided, in various aspects, in unit dosage form for ease of administration and uniformity of dosage. “Unit dosage form” as used herein refers to physically discrete units suited as unitary dosages for the subject to be treated, each unit containing a predetermined quantity of active compound calculated to produce the desired therapeutic effect in association with the required pharmaceutical carrier. The specification for the unit dosage forms are dictated by, and are directly dependent on, the unique characteristics of the excipient(s) and therapeutic agent(s) and the particular biological effect to be achieved.

Safety and efficacy of compositions described herein are determined by standard procedures using in vitro or in vivo technologies, such as the materials and methods described herein. Administration may be on an as-needed or as-desired basis, for example, once-monthly, once-weekly, or daily, including multiple times daily, for example, at least once daily, from one to about ten times daily, from about two to about four times daily, or about three times daily. A dose of composition optionally comprises about from about 0.001 mg to about 1000 mg active agent, alternatively from about 2.5 mg to about 750 mg active agent, and alternatively from about 5 mg to about 650 mg of the active agent. In various embodiments, a dose of composition according to the disclosure comprises 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mg/kg body weight per day.

In various embodiments, a therapeutic agent (e.g., a CypD modulator as described herein), or a pharmaceutical composition comprising a therapeutic agent, is used in combination with one or more other active agents useful for treating or preventing disorders (e.g., obesity and/or kidney indications as described herein). The other active agent(s) can enhance the effects of the therapeutic agent and/or exert other pharmacological effects in addition to those of the therapeutic agent. Non-limiting examples of active agents that can be used in combination with a therapeutic agent are immunosuppressants (e.g., cyclosporine, azathioprine), corticosteroids, anti-inflammatory agents, chemotherapeutic agents, antibiotics, antifungals, antivirals and antiparasitics. As described herein, other exemplary active agents that are contemplated include apoptosis inhibitors (see table 2) that are FDA approved or in clinical trials.

TABLE 2
Pharmacological inhibitors of apoptosis that are FDA approved or in clinical development

| | | | | | |
|---|---|---|---|---|---|
| | Company or | | | | |
| Drug | institution | Type of compound | Target | Status | Indication |

MOMP

| | | | | | |
|---|---|---|---|---|---|
| Minocycline | Danbury Parmacal | Small compound | Mitochondria | Phase I | Huntington disease |
| Rasagiline (Agilect) | Teva | Small compound | Peripheral | FDA approved | Parkinson disease |
| | | | benzodiazepine | | |
| | | | receptor? | | |

p53

| | | | | |
|---|---|---|---|---|
| Amifostine (Ethyol) | Small molecule | p53 | FDA approved | Reduction of renal cisplatin toxicity in |
| | | | | ovarian or non-small cell lung |
| | | | | carcinoma; reduction of radiation |
| | | | | effects on the parotid gland |

Caspases and their endogenous inhibitors

| | | | | | |
|---|---|---|---|---|---|
| IDN-6556 | Pfizer | Small molecule | Caspases | Phase II | Hepatitis C, acute alcoholic hepatitis, |
| liver transplantation | | | | | |
| IDN-6734 | Pfizer | Small molecule | Caspases | Phase I | Acute myocardial infarction |
| VX-740 | Vertex/Aventis | Small molecule | Caspase-1 | Phase II | Rheumatoid arthritis |

Death receptors and their ligands

| | | | | | |
|---|---|---|---|---|---|
| Adalimumab (HUMIRA) | Abbott | Neutralizing mAb | TNF-α | FDA approved | Rheumatoid arthritis, psoriasis, |
| | | | | | ankylosing spondylitis, Crohn disease |
| Infliximab (Remicade) | Centocor/ | Neutralizing mAb | TNF-α | FDA approved | Rheumatoid arthritis, Crohn disease |
| | Schering-Plough | | | | |
| Etanercept (Enbrel) | Amgen/Wyeth | TNFR2/IgG fusion | TNF-α | FDA approved | Rheumatoid arthritis, Crohn disease |
| | | protein | | | |
| ISIS 104828 | Isis | Antisense | TNF-α | Phase II | Rheumatoid arthritis, Crohn disease, |
| | | oligonucleotide | | | psoriasis |

PARP

| | | | | | |
|---|---|---|---|---|---|
| INO-1001 | Inotek | Small molecule | PARP | Phase I | Ischemia/reperfusion damage |
| Nicotine amide | Johns Hopkins | Small molecule | PARP | Phase I | Ataxia telangiectasia |
| | University | | | | |

Antioxidants

| | | | | | |
|---|---|---|---|---|---|
| Adarvone | Mitsubishi-Tokyo | Small molecule | ROS | Phase III; approved in | Reperfusion injury after acute |
| | | | | Japan for treatment of | myocardial infarction |
| | | | | stroke | |
| Idebenone | | Small molecule | ROS | Phase I | Friedreich ataxia |

Kinase inhibitors

| | | | | | |
|---|---|---|---|---|---|
| CEP-1347 | Cephalon/ | Small molecule | MLK inhibitor | Phase II/III | Parkinson disease |
| | H.Lundbeck | | | | |

Table 2-Adapted from Douglas R. Green et al., J Clin Invest. 2005; 115: 2610-2617.
For treatment of obesity, drugs that may be included are orlistat and sibutramine as these are the two medicines approved by the FDA for long-term treatment of obesity. In addition, agents such as zonisamide and topiramate may be used because each has shown some promise as a weight loss agent in specific clinical circumstances. The endocannabinoid receptor antagonist, rimonabant, which is currently in phase III clinical trials, is also contemplated for use in the methods of the disclosure.

For treatment of fibrosis, angiotensin-converting enzyme inhibitors and angiotensin receptor blockers, as well as aldosterone antagonists may be included with the CypD modulator in the pharmaceutical compositions being administered. Renin inhibitor aliskiren and statins (HMG-CoA reductase inhibitors) for reduction of circulating levels of lipids are also contemplated. Pirfenidone (5-methyl-1-phenyl-2(1H)-pyridone), an oral drug with potent antifibrotic properties that may block TGF-β expression and activity as well as ruboxistaurin, a specific inhibitor of PKC-β, is also contemplated for use with a CypD modulator.

To achieve a desired therapeutic outcome in a combination therapy, a therapeutic agent such as a CypD modulator and the other active agent(s) are generally administered to a subject in a combined amount effective to produce the desired therapeutic outcome (e.g., reduction or elimination of one or more symptoms). The combination therapy can involve administering the CypD modulator and the other active agent(s) at about the same time. Simultaneous administration can be achieved by administering a single composition that contains both the CypD modulator and the other active agent(s). Alternatively, the other active agent(s) can be taken separately at about the same time as a pharmaceutical formulation comprising the CypD modulator.

In other alternatives, administration of the therapeutic agent such as a CypD modulator can precede or follow administration of the other active agent(s) by an interval ranging from minutes to hours. In embodiments where the CypD modulator and the other active agent(s) are administered at different times, the CypD modulator and the other active agent(s) are administered within an appropriate time of one another so that both the CypD modulator and the other active agent(s) can exert a beneficial effect (e.g., synergistically or additively) on the recipient. In some embodiments, the CypD modulator is administered to the subject within about 0.5-12 hours (before or after), or within about 0.5-6 hours (before or after), of administration of the other active agent(s). In certain embodiments, the CypD modulator is administered to the subject within about 0.5 hour or 1 hour (before or after) of administration of the other active agent(s).

The following enumerated paragraphs (numbered 1-45) describe exemplary embodiments of the subject matter of the disclosure.

1. A modulator of cyclophilin D biological activity for use in the treatment of a subject having a disorder selected from the group consisting of obesity and kidney disease.

2. The modulator according to paragraph 1 wherein the disorder is obesity.

3. The modulator according to paragraph 2 wherein the obesity is high-fat-diet-induced obesity.

4. The modulator according to paragraph 1 wherein the disorder is kidney disease.

5. The modulator according to paragraph 4 wherein the kidney disease is selected from the group consisting of renal fibrosis, nephrotoxicity, obstructive nephropathy, cisplatin-induced renal disease, acute renal failure, chronic renal failure, renal glomerulopathy and end-stage renal disease.

6. The modulator according to any of paragraphs 1-5 wherein the subject is human.

7. The modulator according to any of paragraphs 1-6 wherein the modulator alters the expression level of a cyclophilin D coding region, thereby modulating cyclophilin D biological activity.

8. The modulator according to any of paragraphs 1-7 wherein the modulator decreases cyclophilin D biological activity.

9. The modulator according to any of paragraphs 1-8 wherein the modulator is selected from the group consisting of siRNA, shRNA, miRNA, antisense nucleic acid and triplex-forming nucleic acid.

10. The modulator according to any of paragraphs 1-8 wherein the modulator is selected from the group consisting of an antibody, a peptide, a non-toxic small molecule, cyclosporine A, a cyclosporine A derivative, a cyclosporine A analog, a NICAM and a quinoxaline derivative.

11. The modulator according to any of paragraphs 1-8 and 10 wherein the modulator is selected from the group consisting of 2,3-di(furan-2-yl)-6-ethoxycarbonylaminoquinoxaline, 2,3-di(furan-2-yl)-6-N—(N′,N′-diethylcarbamoyl)aminoquinoxaline, 2,3-di(furan-2-yl)-6-((R)-3-ethyoxycarbonyl-piperidino) carbonylaminoquinoxaline, 2,3-di(furan-2-yl)-6-((S)-3-ethyoxycarbonyl-piperidino) carbonylaminoquinoxaline, 2,3-di(furan-2-yl)-6-(pyrrolidin-1-yl) carbonylaminoquinoxaline, 2,3-di(furan-2-yl)-6-morpholinocarbonylaminoquinoxaline, 2,3-di(furan-2-yl)-6-N—(N′,N′-diisopropylcarbamoyl)aminoquinoxaline, Debio 025, N-methyl-4-isoleucine-cyclosporin (NIM811), N-methyl-4-valine-cyclosporin (PKF220-384), Antamanide, SCY-635, a polyketide, 3S-[3R*[E(1S*,3S*,4S*)],4S*,5R*,8S*,9E,12R*,14R*,15S*,16R*,18S*,19S*,26aR*-5,6,8,11,12,13,14,15,16,17,18,19,24,25,26,26a-hexadecahydro-5,19-dihydroxy-3-[2-(4-hydroxy-3-methoxycyclohexyl)-1-methylethenyl]-14,16-dimethoxy-4,10,12,18-tetramethyl-8-(2-propenyl)-15,19-epoxy-3H-pyrido[2,1-c][1,4]oxaazacyclotricosine-1,7,20,21(4H,23H)-tetrone monohydrate (FK-506, tacrolimus), L-685,818 (C18-OH, C21-ethyl-FK-506), NeuroSTAT®, CicloMulsion®, ascomycin (FK-520, L-683,590), [32-Arg] ascomycin, [U-13C] ascomycin, dihydro-FK-506 (FK-506D), C18-OH-ascomycin, A-119,435, U-13C-ascomycin, Leu-Ile,N-(glyoxyl)pipecolate ester, L-732,531, L-688,617, CaN A467-492, mitochondrial-targeted cyclosporine A (mtCsA), and Heat Shock Protein 60.

12. The modulator according to any of paragraphs 1-8 wherein the modulator is cyclosporine A.

13. The modulator according to any of paragraphs 1-12 wherein the modulator is in emulsified form.

14. The modulator according to paragraph 13 wherein the modulator is NeuroSTAT®.

15. The modulator according to any of paragraphs 1-3 and 13-14 wherein the modulator is in a composition further comprising a second obesity therapeutic selected from the group consisting of zonisamide, topiramate and rimonabant.

16. The modulator according to any of paragraphs 1 and 4-14 wherein the modulator is in a composition further comprising a second kidney disease therapeutic selected from the group consisting of Minocycline, Rasagiline, Amifostine, IDN-6556 IDN-6734, VX-740, Adalimumab, Infliximab, Etanercept, ISIS 104828, INO-1001, nicotine amide, Edaravone, Idebenone, CEP-1347 and an apoptosis inhibitor.

17. A method of preventing or treating a disorder in a subject wherein the disorder is selected from the group consisting of obesity and kidney disease comprising administering an effective amount of a composition comprising a modulator according to any of paragraphs 1-16.

18. The method according to paragraph 17 wherein the disorder is obesity.

19. The method according to paragraph 18 wherein the obesity is high-fat-diet-induced obesity.

20. The method according to paragraph 17 wherein the disorder is kidney disease.

21. The method according to paragraph 20 wherein the kidney disease is selected from the group consisting of renal fibrosis, nephrotoxicity, obstructive nephropathy, cisplatin-induced renal disease, acute renal failure, chronic renal failure, renal glomerulopathy and end-stage renal disease.

22. The method according to any of paragraphs 17-21 wherein the subject is human.

23. The method according to any of paragraphs 17-22 wherein the modulator alters the expression level of a cyclophilin D coding region, thereby modulating cyclophilin D biological activity.

24. The method according to any of paragraphs 17-23 wherein the modulator decreases cyclophilin D biological activity.

25. The method according to any of paragraphs 17-24 wherein the modulator is selected from the group consisting of siRNA, shRNA, miRNA, antisense nucleic acid and triplex-forming nucleic acid.

26. The method according to any of paragraphs 17-24 wherein the modulator is selected from the group consisting of an antibody, a peptide, a non-toxic small molecule, cyclosporine A, a cyclosporine A derivative, a cyclosporine A analog, a NICAM and a quinoxaline derivative.

27. The method according to any of paragraphs 17-24 and 26 wherein the modulator is selected from the group consisting of 2,3-di(furan-2-yl)-6-ethoxycarbonylaminoquinoxaline, 2,3-di(furan-2-yl)-6-N—(N′,N′-diethylcarbamoyl)aminoquinoxaline, 2,3-di(furan-2-yl)-6-((R)-3-ethyoxycarbonyl-piperidino) carbonylaminoquinoxaline, 2,3-di(furan-2-yl)-6-((S)-3-ethyoxycarbonyl-piperidino) carbonylaminoquinoxaline, 2,3-di(furan-2-yl)-6-(pyrrolidin-1-yl) carbonylaminoquinoxaline, 2,3-di(furan-2-yl)-6-morpholinocarbonylaminoquinoxaline, 2,3-di(furan-2-yl)-6-N—(N′,N′-diisopropylcarbamoyl)aminoquinoxaline, Debio 025, N-methyl-4-isoleucine-cyclosporin (NIM811), N-methyl-4-valine-cyclosporin (PKF220-384), Antamanide, SCY-635, a polyketide, 3S-[3R*[E(1S*,3S*,4S*)],4S*,5R*,8S*,9E,12R*,14R*,15S*,16R*,18S*,19S*,26aR*-5,6,8,11,12,13,14,15,16,17,18,19,24,25,26,26a-hexadecahydro-5,19-dihydroxy-3-[2-(4-hydroxy-3-methoxycyclohexyl)-1-methylethenyl]-14,16-dimethoxy-4,10,12,18-tetramethyl-8-(2-propenyl)-15,19-epoxy-3H-pyrido[2,1-c][1,4]oxaazacyclotricosine-1,7,20,21(4H,23H)-tetrone monohydrate (FK-506, tacrolimus), L-685,818 (C18-OH, C21-ethyl-FK-506), NeuroSTAT®, CicloMulsion®, ascomycin (FK-520, L-683,590), [32-Arg] ascomycin, [U-13C] ascomycin, dihydro-FK-506 (FK-506D), C18-OH-ascomycin, A-119,435, U-13C-ascomycin, Leu-Ile,N-(glyoxyl)pipecolate ester, L-732,531, L-688,617, CaN A467-492, mitochondrial-targeted cyclosporine A (mtCsA) and Heat Shock Protein 60.

28. The method according to any of paragraphs 17-22 and 20-24 wherein the modulator is cyclosporine A.

29. The method according to any of paragraphs 17-28 wherein the modulator is in emulsified form.

30. The method according to paragraph 29 wherein the modulator is NeuroSTAT®.

31. The method according to any of paragraphs 17-19 and 29 wherein the modulator is in a composition further comprising a second obesity therapeutic selected from the group consisting of zonisamide, topiramate and rimonabant.

32. The method according to any of paragraphs 17 and 20-29 wherein the modulator is in a composition further comprising a second kidney disease therapeutic selected from the group consisting of Minocycline, Rasagiline, Amifostine, IDN-6556 IDN-6734, VX-740, Adalimumab, Infliximab, Etanercept, ISIS 104828, INO-1001, nicotine amide, Edaravone, Idebenone, CEP-1347 and an apoptosis inhibitor.

33. A composition comprising a modulator of cyclophilin D activity and a pharmaceutically acceptable carrier formulated for administration to treat or prevent a disorder selected from the group consisting of obesity and kidney disease.

34. The composition according to paragraph 33 wherein the treatment or prevention is the treatment or prevention of obesity.

35. The composition according to paragraph 33 wherein the treatment or prevention is the treatment or prevention of a kidney disorder.

36. The composition according to paragraph 35 wherein for the disorder is selected from the group consisting of renal fibrosis, nephrotoxicity, obstructive nephropathy, cisplatin-induced renal disease, acute renal failure, chronic renal failure, renal glomerulopathy and end-stage renal disease.

37. The composition according to paragraph 33 wherein the modulator is selected from the group consisting of siRNA, shRNA, miRNA, antisense nucleic acid and triplex-forming nucleic acid.

38. The composition according to paragraph 33 wherein the modulator is selected from the group consisting of an antibody, a peptide, a non-toxic small molecule, cyclosporine A, a cyclosporine A derivative, a cyclosporine A analog, a NICAM and a quinoxaline derivative.

39. The composition according to paragraph 33 wherein the modulator is selected from the group consisting of 2,3-di(furan-2-yl)-6-ethoxycarbonylaminoquinoxaline, 2,3-di(furan-2-yl)-6-N—(N′,N′-diethylcarbamoyl)aminoquinoxaline, 2,3-di(furan-2-yl)-6-(R)-3-ethyoxycarbonyl-piperidino) carbonylaminoquinoxaline, 2,3-di(furan-2-yl)-6-(S)-3-ethyoxycarbonyl-piperidino) carbonylaminoquinoxaline, 2,3-di(furan-2-yl)-6-(pyrrolidin-1-yl) carbonylaminoquinoxaline, 2,3-di(furan-2-yl)-6-morpholinocarbonylaminoquinoxaline, 2,3-di(furan-2-yl)-6-N—(N % N′-diisopropylcarbamoyl)aminoquinoxaline, Debio 025, N-methyl-4-isoleucine-cyclosporin (NIM811), N-methyl-4-valine-cyclosporin (PKF220-384), Antamanide, SCY-635, a polyketide, 3S-[3R*[E(1S*,3S*,4S*)],4S*,5R*,8S*,9E,12R*,14R*,15S*,16R*,18S*,19S*,26aR*-5,6,8,11,12,13,14,15,16,17,18,19,24,25,26,26a-hexadecahydro-5,19-dihydroxy-3-[2-(4-hydroxy-3-methoxycyclohexyl)-1-methylethenyl]-14,16-dimethoxy-4,10,12,18-tetramethyl-8-(2-propenyl)-15,19-epoxy-3H-pyrido[2,1-c][1,4]oxaazacyclotricosine-1,7,20,21(4H,23H)-tetrone monohydrate (FK-506, tacrolimus), L-685,818 (C18-OH, C21-ethyl-FK-506), NeuroSTAT®, CicloMulsion®, ascomycin (FK-520, L-683,590), [32-Arg] ascomycin, [U-13C] ascomycin, dihydro-FK-506 (FK-506D), C18-OH-ascomycin, A-119,435, U-13C-ascomycin, Leu-Ile,N-(glyoxyl)pipecolate ester, L-732,531, L-688,617, CaN A467-492, mitochondrial-targeted cyclosporine A (mtCsA) and Heat Shock Protein 60.

40. The composition according to paragraph 33 wherein the modulator is cyclosporine A.

41. The composition according to paragraph 33 wherein the modulator is in emulsified form.

42. The composition according to paragraph 41 wherein the modulator is NeuroSTAT®.

43. The composition according to paragraph 34 wherein the modulator is in a composition further comprising a second obesity therapeutic selected from the group consisting of zonisamide, topiramate and rimonabant.

44. The composition according to paragraph 35 wherein the modulator is in a composition further comprising a second kidney disease therapeutic selected from the group consisting of Minocycline, Rasagiline, Amifostine, IDN-6556 IDN-6734, VX-740, Adalimumab, Infliximab, Etanercept, ISIS 104828, INO-1001, nicotine amide, Edaravone, Idebenone, CEP-1347 and an apoptosis inhibitor.

45. A kit for treating a disorder selected from the group consisting of obesity and kidney disease comprising a therapeutically effective amount of a modulator of cyclophilin D, a pharmaceutically acceptable carrier and instructions for use thereof in treating the disorder.

The disclosure is further described in the following examples. The examples serve only to illustrate the disclosure and are not intended to limit the scope of the disclosure in any way.

### Example 1

### Cyclophilin D Deficiency Prevents Diet-Induced Obesity

Mitochondrial coupling efficiency is pivotal in thermogenesis and energy homeostasis. The data disclosed in this Example establish that deletion of cyclophilin D (CypD), a key modulator of the mitochondrial permeability transition pore, led to resistance to diet-induced obesity (DIO) in both male and female mice, due to increased basal metabolic rate, heat production, total energy expenditure and expenditure of fat energy, despite increased food consumption. Absorption of fatty acids is not altered between CypD−/− and wild-type (WT) mice. Adult CypD−/− mice developed hyperglycemia, insulin resistance and glucose intolerance, albeit resistant to DIO. These data demonstrate that inhibition of CypD function could protect from high-fat diet-induced obesity (HFDIO) by increasing energy expenditure in both male and female mice. Inhibition of CypD thus offers a novel target to modulate metabolism and prevent or treat obesity.

I. Materials and Methods

A. Animal Care and Feeding Studies.

Breeding pairs of CypD−/− mice were purchased from Jackson Laboratories, Maine. CypD−/− mice are viable, fertile, normal in size and do not display any gross physical or behavioral abnormalities. The respective WT control mice for CypD−/− mice (B6129SF2/J) were purchased from Jackson Laboratories. Thirty-five-to-forty-day-old male and female mice were maintained on a 12-hour light/dark cycle with free access to either a control-diet (10% fat by calories, 3.85 kcal/g), or a HF diet (45% fat by calories, 4.76 kcal/g, Research diets, Inc.). Food intake and body weight were measured weekly. All Animal studies and procedures were approved by the institutional animal care and usage committee.

B. Body Composition Analysis by NMR Method.

At the end of the long-term feeding study (19 weeks on the high-fat (HF) diet), the body compositions of CypD^{-/-/-} and WT mice were analyzed with a commercial nuclear magnetic resonance machine (EchoMRI-100; Echo Medical Systems). Body fat and lean tissue were measured during the test.

C. Energy Expenditure.

On the same group of animals indicated above, oxygen consumption and carbon dioxide production were measured using an indirect calorimeter (AccuScan Instruments Inc.). Constant airflow (0.75 liters/minute) was drawn through the chamber and monitored by a mass-sensitive flowmeter. Properties assessed with this measurement include VO₂, VCO₂, RQ, and HEAT [Strader, A. D., et al., J. Clin. Invest., 114: 1354-60 (2004)]. To calculate oxygen consumption (VO₂), carbon dioxide production (VCO₂), and respiratory quotient (RQ; ratio of VCO₂ to VO₂), gas concentrations were monitored at the inlet and outlet of the sealed chambers.

D. Temperature Measurements.

Body temperatures were measured using a rectal digital thermometer probe (model 4600; Yellow Springs Instruments, Yellow Springs, Ohio).

E. Measurement of Blood Glucose.

Blood was collected from fasted (12 hours) mice at various time points and different assays were performed as indicated herein. Blood glucose was assayed with a glucometer (Hemocue glucose 201 analyzer).

F. Intraperitoneal Glucose Tolerance Test (IPGTT).

At the end of the long-term feeding study (19 weeks on the HF diet), 5-hour fasted mice were injected intraperitoneally with D-glucose (30% solution; 2 mg/g of body weight), and blood glucose values were determined at 0, 15, 30, 60, and 120 minutes post-injection. Baseline and test samples were all obtained from the tail vein [Strader, A. D., et al., J. Clin. Invest., 114: 1354-60 (2004)].

G. Insulin Tolerance Tests (ITT).

ITT were performed on the same group of animals 10 days after the IPGTT test. Mice were fasted for 5 hours and blood was collected for baseline glucose values. Animals were injected intraperitoneally with 0.5 mU/g insulin (100 mU/ml solution), and blood glucose values were determined at 0, 15, 30 and 60 minutes post-injection.

H. Statistical Analysis.

Data from long-term body weight studies and ITT and IPGTT were analyzed by two-way analysis of variance (ANOVA) with repeated measures. For comparisons of body composition measures, food intake (kcal), mean kcal/hour, and fasting plasma glucose, a Student's t test was utilized. P values less than 0.05 were considered statistically significant. All values are presented as means±SEM unless otherwise specified.

II. Results

A. CypD Deficient Mice are Resistant to High Fat Diet-Induced Obesity.

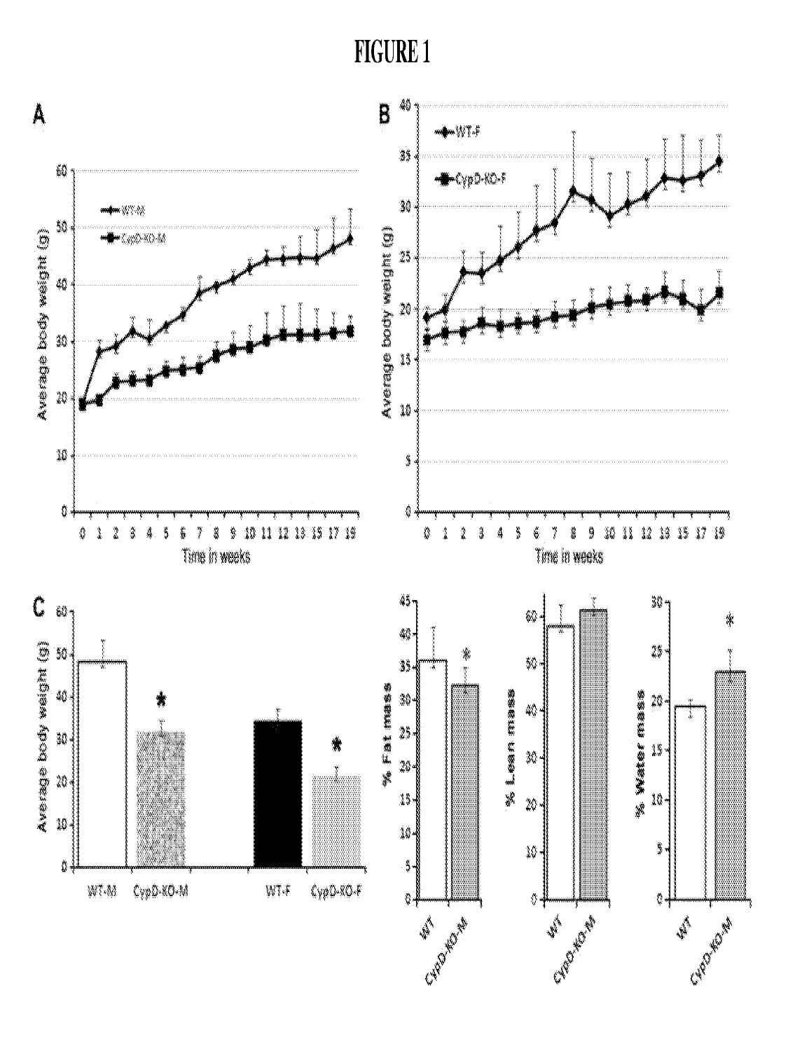

In order to determine the effect of CypD deficiency on energy homeostasis, six-week-old male and female cypD^{-/-} mice along with age-matched and genetically matched wild-type (B6129SF2/J) mice were fed with either high fat (HF; 45% fat by calories, 4.76 kcal/g) or normal diet (ND; 10% fat by calories, 3.85 kcal/g) for a period of 19 weeks. The body weights of mice from different groups were similar at the beginning of the study. Mice from all groups were weighed weekly and quantitative data demonstrated that the rate at which body weight increased was significantly lower in HF-fed CypD^{-/-} male (FIG. 1A) and female (FIG. 1B) mice compared to their wild-type (WT) counterparts. CypD^{-/-} mice appeared smaller in size compared to their WT counterparts at 17 weeks post-HF diet. These results demonstrate that both male and female cypD^{-/-} mice are resistant to HF diet-induced obesity.

To understand the mechanism by which CypD deficiency protects against obesity, the body weight and fat mass, food intake, metabolic level and energy expenditure were measured in CypD^{-/-} and WT mice. At 19 weeks post-HF feeding, the difference in body weight between cypD^{-/-} and WT male mice was 34.6% (n=4; p<0.01) and female mice was 38.4% (FIG. 1C). No difference in body weight between control-diet-fed WT and CypD^{-/-} mice was noted up to 19 weeks post-feeding. The body compositions of cypD^{-/-} and WT male mice fed HF was analyzed by quantitative nuclear magnetic resonance (NMR) at the 19-week study's completion time point. The data revealed that CypD^{-/-} mice have a significantly lower percentage of fat mass (p<0.01; n=4), increased water content (p<0.01; n=4), but no change in the percentage of lean mass, compared to WT mice (FIG. 1C).

B. Food Consumption is Decreased in Female but not in Male cypD^{-/-} Mice.

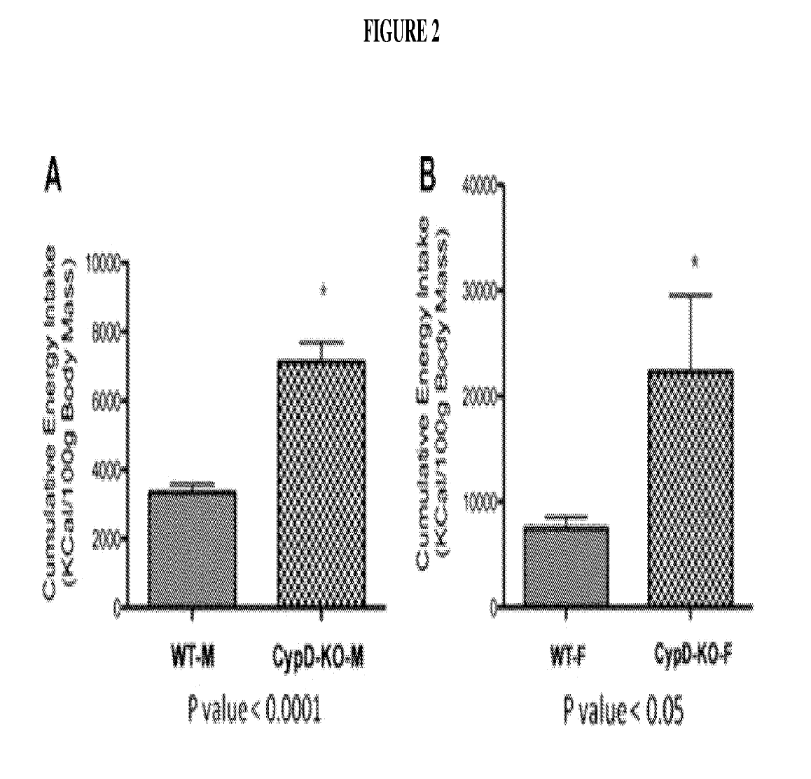

Measurement of food intake in HF-fed CypD^{-/-} and WT male mice demonstrated that the cumulative food intake over a period of 17 weeks, adjusted for body mass, demonstrated a significant increase in both male and female CypD^{-/-} mice (FIG. 2A and FIG. 2B, respectively). The average feed consumption in absolute mass and kilocalories, however, are comparable during the high-fat feeding period in male CypD^{-/-} and WT mice, but significantly decreased in female CypD^{-/-} mice on the HF diet compared to their WT counterparts. The cumulative feed efficiency (increase in body weight relative to energy intake), determined as previously described [Zigman, J. M. et al., J. Clin. Invest., 115: 3564-72 (2005), incorporated herein by reference in its entirety] was also significantly decreased in both male and female CypD^{-/-} mice compared to their WT counterparts. These data indicate that the decrease in body weight in CypD^{-/-} male mice is independent of feed intake and, despite similar levels of feed consumption, CypD^{-/-} male mice did not accumulate fat, indicating an increased expenditure of energy, possibly by the uncoupling of respiration.

C. Food Reabsorption is not Altered in CypD-KO Mice.

In order to determine if the difference in fat absorption efficiency between WT and CypD-KO mice was due to a difference in fat reabsorption, fecal lipid output was measured directly after mice were fed the HF diet for 18 weeks. The percentage of palmitate (16:0) in the fecal sample was 34.86±2.358 and 27.14±3.470 (p>0.05; n=4) in CypD-KO and WT mice, respectively. The percentage of stearate (18:0) in the fecal sample was 56.59±4.488 and 50.22±4.081; (p>0.05; n=4) in WT and CypD-KO mice, respectively. The percentage of oleate (18:1) in the fecal sample was 11.86±4.488 and 12.75±4.081; (p>0.05; n=4) in WT and CypD-KO mice, respectively. These data demonstrate that there are no significant changes in absorption of the fat content as the fecal content of fat was not altered between the groups. Thus, the decreased obesity in CypD-KO mice is not due to an alteration in fecal lipid output when the animals are maintained on a high-fat diet. Collectively, these data indicate that the resistance to DIO observed in HF-fed CypD^{-/-} mice is not due to hypophagia or an alteration in fecal lipid output.

D. Decreased Body Weight in cypD^{-/-} Mice is Partly Due to Increased Energy Expenditure.

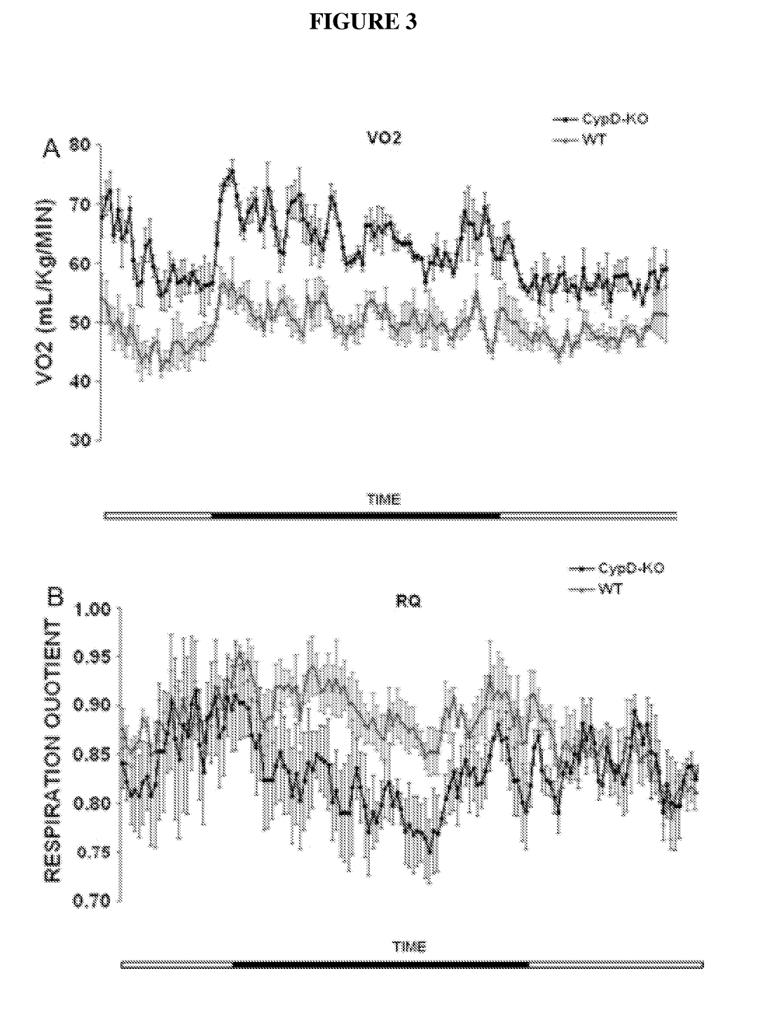

Indirect calorimetry experiments were carried out to investigate whether the resistance to weight gain in CypD^{-/-} mice is due to increased energy expenditure. CypD^{-/-} mice maintained on a HF diet for 19 weeks were placed in the indirect calorimeter for 24 hours with unlimited access to HF diet and water. CypD^{-/-} mice consistently had an increase in metabolic rate, as demonstrated by an increase in oxygen consumption, compared to WT mice (61.93±0.35 in CypD^{-/-} versus 49.39 liter/kg of body weight of VO₂ in WT mice) (FIG. 3A) as well as an increase in CO₂ produced. The respiratory quotient of 0.82 measured during the active period (dark cycle) in CypD^{-/-} mice indicated that these mice used fatty acids as the main energy source while a respiratory quotient of 0.89 in WT mice indicated the use of more carbohydrates than fat as their energy source (FIG. 3B). Thus, the effect of CypD deficiency on decreasing body weight and fat mass may be influenced by the decreased respiratory quotient and increase in fat oxidation. This position is further supported by the observation that levels of circulating triglycerides and free fatty acids were similar in both genotypes, suggesting that in CypD^{-/-} mice, triglycerides were being rapidly oxidized in energy-consuming tissues. Further, NMR analysis of the body composition of cypD^{-/-} and WT male mice fed HF also revealed that CypD^{-/-} mice have a significantly lower percentage of fat mass. Heat (cal/hour/kg) was measured for 24 hours a in cypD^{-/-} and WT mice during the 24-hour period (FIG. 3C) and was 18144 cal/kg/hour and 14572 cal/kg/hour, respectively. The mean energy expenditure (EE) was calculated separately during both light and dark cycles. CypD^{-/-} mice demonstrated increased energy expenditure during both cycles.

E. Body Temperature.

The body temperature of WT and CypD^{-/-} mice was measured using a rectal probe. Heat production (calories/hour/kg) is a measure of caloric output and is directly proportional to metabolic rate. Both WT and CypD^{-/-} male mice displayed the expected diurnal rhythm with increased heat production during the dark period (p<0.05). Consistent with an increase in energy expenditure (EE), female CypD^{-/-} mice had a higher core body temperature. (38.58±0.54) compared to female WT mice (38.025±0.44; n=4; p=0.181) and male CypD^{-/-} mice had a higher core body temperature (38.158±0.54) compared to male WT mice (37.58±0.44; n=4), although the increase was not statistically significant. The increase in body temperature of female KO mice was more prominent, a finding consistent with their complete resistance to weight gain post-HF feeding. The higher core body temperature found in CypD^{-/-} mice indicated that thermogenic mechanisms are more active in these mice to maintain homeothermy.

F. Normal Glucose Levels in cypD^{-/-} Mice During HF Feeding.

Glucose levels were monitored in CypD^{-/-} mice and WT mice at several time points post-HF feeding. In both WT and CypD^{-/-} mice, the levels increased gradually but were not different during the 19-week period of the study.

G. Lack of Insulin Sensitivity and Impaired Glucose Tolerance in CypD^{-/-} HF-Fed Mice.

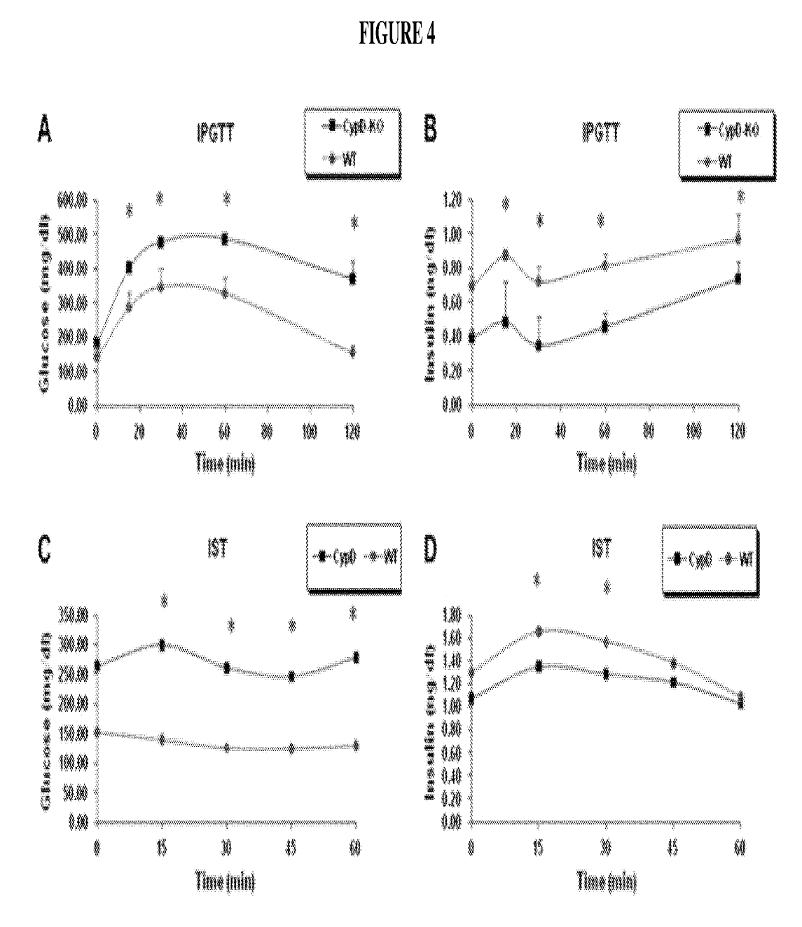

In order to determine if there were any alterations in glucose homeostasis in CypD^{-/-} mice, an intraperitoneal glucose tolerance test (IPGTT) was performed. WT mice and CypD^{-/-} mice that were fed HF-diet for 19 weeks were fasted for 5 hours followed by intraperitoneal injection with 20% D-glucose. Tail blood was collected for measurement of glucose and insulin. Intriguingly, CypD^{-/-} mice had decreased ability for glucose clearance as the serum glucose levels were significantly higher over a 120-minute period. Serum glucose levels were elevated at 15 minutes and reached even higher levels at later time points. The average values of glucose in mg/dL in WT were 144, 288, 347, 329 and 156 and in CypD^{-/-} mice were 182, 402, 478, 487 and 370 at 0-, 15-, 30-, 60- and 120-minutes post-glucose administration (FIG. 4A). In a separate IPGTT test, insulin measurements were performed over the 120-minute post-glucose administration period. Interestingly, the plasma insulin levels were significantly lower in CypD^{-/-} mice 0.392, 0.484, 0.348, 0.455 and 0.739 ng/mL in relation to WT mice 0.696, 0.875, 0.0.724, 0.0.817 and 0.973 ng/mL at 0-, 15-, 30-, 60- and 120-minutes post-glucose administration (FIG. 4B). The decrease in insulin levels observed in CypD^{-/-} mice, compared to WT mice at 19 weeks post-HF feeding, indicates that insulin production, secretion, and its maturation process may be impaired or, alternatively, its degradation process may be accelerated in CypD^{-/-} mice.

The ability of insulin to acutely moderate the levels of glucose or glucose clearance was assessed by performing an insulin tolerance test (ITT). At 19 weeks post-HF feeding, the ability of insulin to acutely stimulate glucose disposal in CypD^{-/-} mice was significantly blunted throughout the 60-minute period monitored, indicating decreased insulin sensitivity or insulin availability (FIG. 4C). The level of glucose was 278 mg/dL in CypD^{-/-} mice compared to 129 in WT mice (p<0.05; n=4 per group) at the 60-minute time point post-insulin administration. The amount of serum insulin post-IST test shows that the level of insulin in CypD^{-/-} mice is significantly lower compared to that in WT mice at 15- and 30-minute time periods post-insulin administration (FIG. 4D). These data indicate that insulin is removed from circulation at a higher pace in CypD^{-/-} mice compared to that in WT mice.

H. Cyclosporine A, an Inhibitor of CypD, Prevents Diet-Induced Obesity

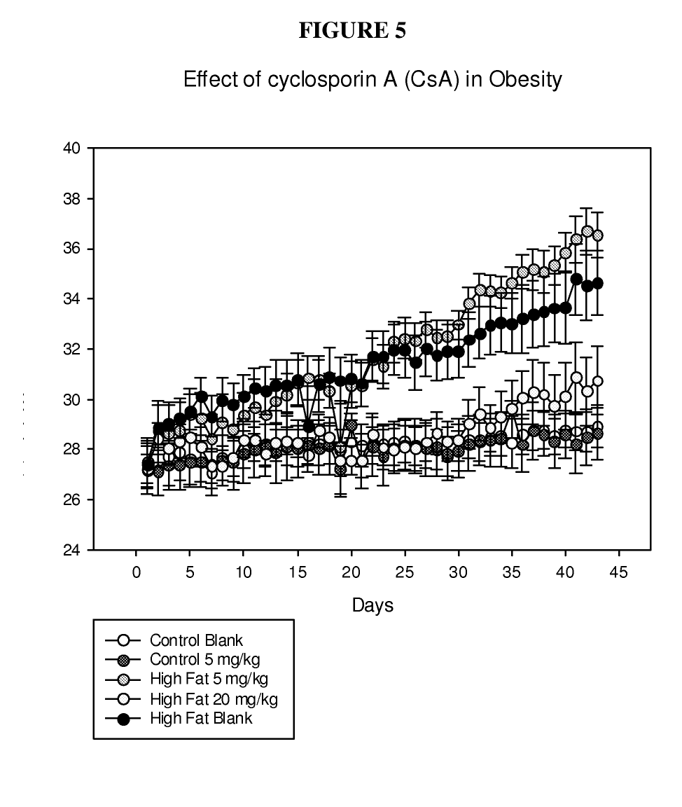

As shown in FIG. 5, administration of cyclosporine A (CsA) prevented obesity development. Wild-type (C57Bl/6) mice were divided into 5 groups and were fed either a normal diet or a high-fat diet. The mice were treated with blank, 5 mg/kg/day, or 20 mg/kg/day of the Neurovive® drug (emulsified cyclosporine A) for 23 days. The drug as well as the blank was withdrawn at day 23 and the mice were allowed to continue on their respective diets for another 20 days. The effect of drug treatment and its withdrawal on the development of obesity is shown in FIG. 5.

The data in FIG. 5 establish that inhibition of cypD using the drug inhibited obesity development. These data concur with data from the CypD gene ablation model, further supporting a role for CypD in obesity development, as well as confirming the use of the drug in obesity treatment.

A major factor that could contribute to the decreased body weight after high-calorie intake is the imbalance in energy intake and expenditure [Spiegelman, B. M., et al., Cell, 104: 531-43 (2001)]. The quantitative data presented here demonstrate that male HF-fed CypD−/− mice have similar feed intake, feed efficiencies and gross energetic efficiencies compared to their WT sex-matched counterparts. The data presented in this Example indicate that CypD−/− mice waste more of the consumed energy and store less in body energy stores than WT mice. The instant Example demonstrates that there are no significant changes in absorption of fat content as the fecal content of fat was not altered between the groups. Taken together, these findings indicate that an increased energy output in CypD−/− male mice contributes to their resistance to the obesity phenotype.

Concurring with the decreased weight gain observed in HF-fed CypD−/− mice, CypD deficiency increased the oxygen consumption (VO2), as well as the CO2 produced (VCO2) throughout the 24-hour time period that was measured. The alterations in VO2 and in VCO2 resulted in a decreased RQ of 0.76 compared to 0.82 in WT mice. Because about 90% of mammalian VO2 is mitochondrial [Rolfe, D. F. et al., Physiol. Rev., 77: 731-58 (1997)], increased VO2 levels expressed per gram seen in CypD−/− mice are a direct reflection of increased mitochondrial oxidative metabolism. The RQ and VO2 data from this experiment show that fat is more relied upon as a mitochondrial fuel substrate in CypD−/− animals than in their WT controls. Thus, the effect of CypD deficiency on decreasing body weight and fat mass is influenced by the respiratory quotient, the increase in fat oxidation, and physical activity.

In this Example, HF-fed CypD−/− mice had increased energy expenditure (EE) compared to their WT counterparts. EE derives from thermogenesis resulting from cellular metabolic processes, including basal metabolism, adaptive thermogenesis and physical activity. Basal metabolism represents the heat production of the body in a thermoneutral environment, under resting conditions [Rolfe, D. F. et al., Physiol. Rev., 77: 731-58 (1997)]. The core body temperature measurements indicate that basal metabolism is increased in HF-fed CypD−/− male and female mice, consistent with an increase in total EE and increased oxygen consumption. The higher core body temperature found in CypD−/− mice indicates that thermogenic mechanisms are more active in these mice to maintain homeothermy. Because there is a significant energy cost attached to maintenance of body temperature [Silva, J. E., Physiol. Rev., 86: 435-64 (2006)], this at least partially accounts for the increased metabolism seen in CypD−/− mice. Heat production (calories/hour/kg) is a measure of caloric output and is directly proportional to metabolic rate. In a study using male mice, both WT and CypD−/− male mice displayed the expected diurnal rhythm with increased heat production during the dark period (p<0.05). Heat production per animal was increased in CypD−/− male mice compared to WT mice over a 24-hour time period.

Adaptive thermogenesis is the process by which energy is dissipated in the form of heat in response to environmental changes, such as exposure to cold and alterations in diet [Spiegelman, B. M., et al., Cell, 104: 531-43 (2001)]. Under homeostatic conditions, adaptive thermogenesis is a key mechanism by which a body maintains its core temperature and regulates energy expenditure; its dysregulation promotes obesity [Fan, W., et al., Peptides, 26: 1800-13 (2005)]. Exposure of CypD−/− male and female mice to cold temperature did not result in significant changes in their core body temperature over a 6-hour time period, as described in section E, above.

The data in FIG. 5 indicate that inhibition of cypD using NeuroSTAT® (20 mg/kg; Neurovive Pharmaceutical AB) inhibited high fat diet-induced obesity development in mice. These data concur with the data from the CypD gene ablation mouse model, further establishing a role for CypD in obesity development as well as the suitability of CypD modulators for use in preventing or treating obesity.

To conclude, the data presented in this Example indicate that CypD deficiency prevents diet-induced obesity by increasing energy expenditure, indicating that CypD play a central role in mitochondrial bioenergetics and the development of obesity. The data in FIG. 5 indicating that inhibition of cypD using the drug inhibited obesity development further supports this conclusion.

The data disclosed in this Example were obtained using the CypD gene ablation model. One of ordinary skill in the art will understand that these results are obtained using a variety of well-understood methods of reducing or inhibiting the activity of CypD. Exemplary methods of inhibiting CypD activity include, but are not limited to, the use of any form of an anti-CypD antibody, an inhibitory peptide, an inhibitory small molecule, or an inhibitory nucleic acid, such as an siRNA directed against the coding region for CypD (e.g., sc-44892 Cyclophilin D siRNA; Santa Cruz Biotechnology, Inc.). With respect to inhibitory nucleic acids, beyond siRNAs the disclosure contemplates anti-sense nucleic acids such as anti-sense RNA, e.g., at least 10 contiguous nucleotides of the sequence complementary to the coding region of CypD set forth in SEQ ID NO:1, wherein the nucleic acid inhibits CypD expression.

### Example 2

Cyclophilin D Deficiency Prevents Development of Fibrosis and Inflammation Associated with Renal Fibrosis

Renal interstitial fibrosis results from the progression of most forms of renal disease in an irreversible process that eventually leads to end-stage renal disease (ESRD). The presence of interstitial fibrosis, a key structural component of obstructive nephropathy, is the major cause of chronic kidney disease (CKD) in children and young adults. Unilateral ureteral obstruction (UUO), which has become the standard model of renal interstitial fibrosis, results in renal functional loss including decreased renal blood flow and glomerular filtration rate, as well as histological changes including apoptosis and necrosis, tubular atrophy and dilation, leukocyte infiltration, inflammation, and tubulointerstitial extension. Although much progress has been made in understanding the cellular and molecular mechanisms of interstitial fibrosis, there remains a gap in our knowledge of key factors that facilitate fibrogenesis.

Renal tubular apoptosis is a major factor leading to tubular atrophy following unilateral ureteral obstruction. Increased reactive oxygen species, and a renal environment favoring pro-apoptotic, over survival, signals, contribute to cell death. Although necrosis, autophagy and other forms of cell death are not implicated in UUO, it is possible that such forms of cell death may also participate in the injury process. CypD has been shown to participate in apoptosis and necrosis and its inhibition protects from cell death in various stress and disease conditions. It was unknown, however, whether cypD gene ablation would protect kidneys from the injury induced by UUO.

### Materials and Methods

### A. Animal Preparation

Mice were cared before and during the experimental procedures in accordance with the policies of the Institutional Animal Care and Use Committee (IACUC), University of Nebraska Medical Center (UNMC), and the National Institutes of Health Guide for the Care and Use of Laboratory Animals. All protocols had received prior approval from the UNMC-IACUC. CypD-knockout (KO) and wild-type (WT) male mice (8- to 10-week-old; Jackson Laboratories, Bar Harbor, Me.) were anesthetized by intraperitoneal injection of a cocktail containing ketamine (200 mg/kg body weight) and xylazine (16 mg/kg body weight). After that, the left ureter was obstructed completely near the renal pelvis using a 6-0 silk tie. Sham-operated mice underwent the same surgical procedure except for the ureter ligation. After 3 or 10 days of UUO or sham, kidneys were either fixed in 4% paraformaldehyde for histological studies or snap-frozen in liquid nitrogen for biochemical studies. Hematoxylin & eosin (H&E)-stained sections were used for histological damage score.

### B. Collagen Deposition

Collagen deposition was assessed by both Sirius red staining and hydroxyproline assay. In order to assess Sirius red staining, the paraffin section was stained with Sirius red solution (0.1% Direct Red 80 and 1.3% picric acid, Sigma, St. Louis, Mo.) and washed in acidified water (0.5% acetic acid, Sigma). The area of positive Sirius red staining was measured in at least 5 high-power fields (×200 magnification) per kidney using National Institutes of Health Image software (Image J). For the hydroxyproline assay, the kidney was homogenized in 10 N HCl and hydrolyzed by autoclaving. The hydrolysate was incubated in a Chloramine T reagent (0.84% chloramine-T, 42 mM sodium acetate, 2.6 mM citric acid, and 39.5% isopropanol (pH 6.0)). Next, the sample was incubated in a DMAB reagent (15% 4-(Dimethylamino)benzaldehyde in isopropanol/perchloric acid (2:1 v/v)), and absorbance was measured at 550 nm.

### C. Western Blot Analysis

Western blot analysis was conducted using conventional, well-known techniques and using various antibodies against the β-actin protein (Sigma, cat. no. A5316) and against the α-smooth muscle actin protein (α-SMA; Sigma, cat. no. A2548 (in vivo studies) and cat. no. A5228 (in vitro studies)). Band intensities were analyzed by LabWorks software (Ultra-Violet Products, Cambridge, UK).

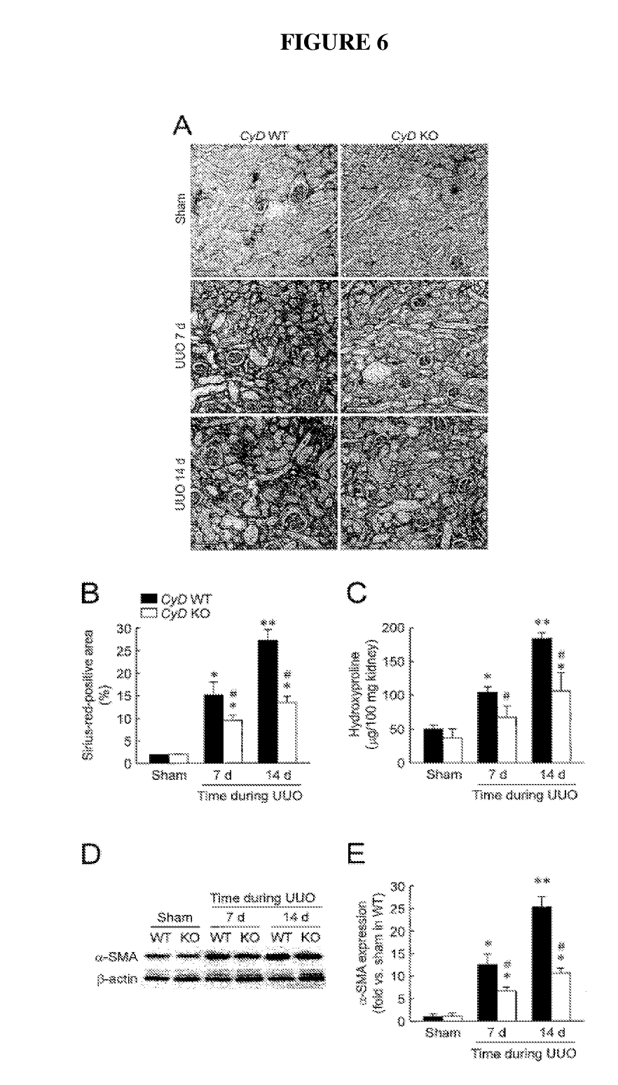

As shown in FIG. 6, CypD deficiency prevented development of fibrosis and inflammation in the well-established unilateral obstruction model of renal fibrosis. CypD deficiency reduced collagen deposition and alpha-smooth muscle actin (α-SMA) expression during unilateral ureteral obstruction, indicating that CypD is required for renal fibrosis in obstructive nephropathy.

### Example 3

### Cyclophilin D Deficiency Prevents Cisplatinum-Induced Renal Toxicity

Cisplatin is one of the most commonly used drugs for the treatment of malignant tumors in testis, ovary, bladder, head and neck, breast and many other tissues/organs. Although effective, the use of cisplatin is limited by its severe side effects in normal tissues. Among these side effects is the major side effect during cisplatin treatment, which is nephrotoxicity. After cisplatin treatment, approximately one-third of patients develop renal dysfunction, resulting in acute renal failure. Kidney tubular cell death is recognized as a major pathogenic factor during cisplatin nephrotoxicity. In cultured kidney tubule epithelial cells in vitro, both apoptotic and necrotic cell death are dependent on cisplatin concentration while, in in vivo animal models, both cell death types are simultaneously induced in kidney tubules after cisplatin injection. Recent studies have focused on the mechanisms of apoptosis induced by cisplatin in kidney tubular cells, and much less on necrotic pathways. Experimental studies indicate a key role of cypD in necrotic cell death. Activation of CypD in the mitochondria by Ca2+ reactive oxygen species are shown to induce the opening of the mitochondrial permeability transition pore (MPTP), mitochondrial swelling, and necrotic cell death. CypD inhibition or gene deletion has been shown to be renoprotective against ischemia-reperfusion injury in the heart, brain and kidney. Since necrosis is a hallmark of cisplatin nephrotoxicity, it was hypothesized that CypD activation may be required for cisplatin nephrotoxicity. To investigate this hypothesis, the cisplatin-induced kidney injury model was employed to assess kidney dysfunction in CypD-KO and wild-type (WT) mice.

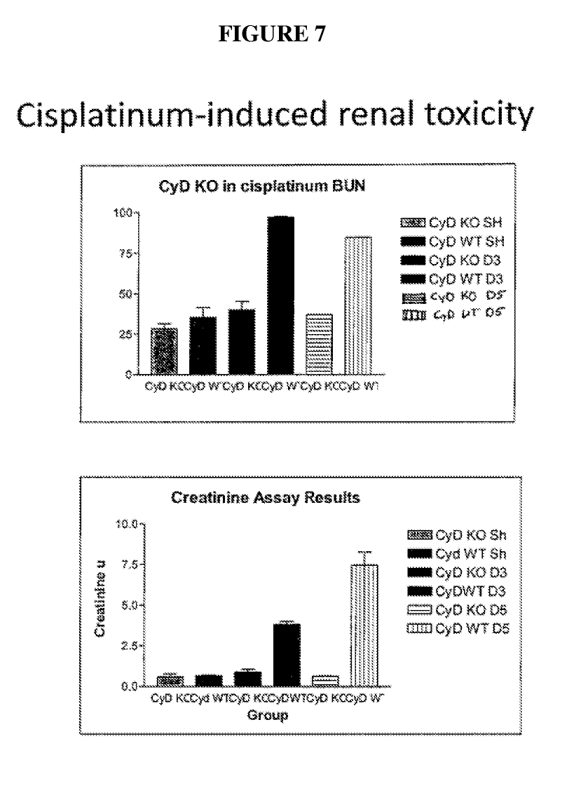

The effect of CypD gene ablation on renal functions after cisplatinum exposure is shown in FIG. 7. Renal function was evaluated by measuring blood urea nitrogen (BUN) levels in the serum at 3 and 5 days post-cisplatinum injury. Renal function was also assessed by measuring serum creatinine levels at 3 and 5 days post-cisplatinum injury. The results establish that CypD gene ablation prevented the increase in both BUN and serum creatinine levels compared to that the levels in WT mice, indicating that kidney damage caused by administration of the anti-cancer agent cisplatin was attenuated in the absence of cypD. These data indicate that inhibition of CypD has a beneficial effect on renal function following cisplatinum exposure, reducing the kidney damage caused by administration of the anti-cancer agent. Thus, inhibition of CypD function(s) provides preventive strategies to combat cisplatin-induced nephrotoxicity.

The disclosed subject matter has been described with reference to various specific and preferred embodiments and techniques. It should be understood, however, that many variations and modifications may be made while remaining within the spirit and scope of the disclosed subject matter.

1. A modulator of cyclophilin D biological activity for use in the treatment of a subject having a disorder selected from the group consisting of obesity and kidney disease.

2. (canceled)

3. The modulator according to claim 1 wherein the obesity is high-fat-diet-induced obesity.

4. (canceled)

5. The modulator according to claim 1 wherein the kidney disease is selected from the group consisting of renal fibrosis, nephrotoxicity, obstructive nephropathy, cisplatin-induced renal disease, acute renal failure, chronic renal failure, renal glomerulopathy and end-stage renal disease.

6. (canceled)

7. The modulator according to claim 1 wherein the modulator alters the expression level of a cyclophilin D coding region, thereby modulating cyclophilin D biological activity.

8. The modulator according to claim 1 wherein the modulator decreases cyclophilin D biological activity.

9. The modulator according to claim 1 wherein the modulator is selected from the group consisting of siRNA, shRNA, miRNA, antisense nucleic acid and triplex-forming nucleic acid.

10. The modulator according to claim 1 wherein the modulator is selected from the group consisting of an antibody, a peptide, a non-toxic small molecule, cyclosporine A, a cyclosporine A derivative, a cyclosporine A analog, a NICAM and a quinoxaline derivative.

11. The modulator according to claim 1 wherein the modulator is selected from the group consisting of 2,3-di(furan-2-yl)-6-ethoxycarbonylaminoquinoxaline, 2,3-di(furan-2-yl)-6-N—(N′,N′-diethylcarbamoyl)aminoquinoxaline, 2,3-di(furan-2-yl)-6-((R)-3-ethyoxycarbonyl-piperidino) carbonylaminoquinoxaline, 2,3-di(furan-2-yl)-6-((S)-3-ethyoxycarbonyl-piperidino) carbonylaminoquinoxaline, 2,3-di(furan-2-yl)-6-(pyrrolidin-1-yl) carbonylaminoquinoxaline, 2,3-di(furan-2-yl)-6-morpholinocarbonylaminoquinoxaline, 2,3-di(furan-2-yl)-6-N—(N′,N′-diisopropylcarbamoyl)aminoquinoxaline, Debio 025, N-methyl-4-isoleucine-cyclosporin (NIM811), N-methyl-4-valine-cyclosporin (PKF220-384), Antamanide, SCY-635, a polyketide, 3S-[3R*[E(1S*,3S*,4S*)],4S*,5R*,8S*,9E,12R*,14R*,15S*,16R*,18S*,19S*,26aR*-5,6,8,11,12,13,14,15,16,17,18,19,24,25,26,26a-hexadecahydro-5,19-dihydroxy-3-[2-(4-hydroxy-3-methoxycyclohexyl)-1-methylethenyl]-14,16-dimethoxy-4,10,12,18-tetramethyl-8-(2-propenyl)-15,19-epoxy-3H-pyrido[2,1-c][1,4]oxaazacyclotricosine-1,7,20,21(4H,23H)-tetrone monohydrate (FK-506, tacrolimus), L-685,818 (C18-OH, C21-ethyl-FK-506), NeuroSTAT®, CicloMulsion®, ascomycin (FK-520, L-683,590), [32-Arg] ascomycin, [U-13C] ascomycin, dihydro-FK-506 (FK-506D), C18-OH-ascomycin, A-119,435, U-13C-ascomycin, Leu-Ile,N-(glyoxyl)pipecolate ester, L-732,531, L-688,617, CaN A467-492, mitochondrial-targeted cyclosporine A (mtCsA) and Heat Shock Protein 60.

12. The modulator according to claim 1 wherein the modulator is cyclosporine A.

13. (canceled)

14. The modulator according to claim 1 wherein the modulator is NeuroSTAT®.

15. The modulator according to claim 1 wherein the modulator is in a composition further comprising a second obesity therapeutic selected from the group consisting of zonisamide, topiramate and rimonabant.

16. The modulator according to claim 1 wherein the modulator is in a composition further comprising a second kidney disease therapeutic selected from the group consisting of Minocycline, Rasagiline, Amifostine, IDN-6556 IDN-6734, VX-740, Adalimumab, Infliximab, Etanercept, ISIS 104828, INO-1001, nicotine amide, Edaravone, Idebenone CEP-1347 and an apoptosis inhibitor.

17. A method of preventing or treating a disorder in a subject wherein the disorder is selected from the group consisting of obesity and kidney disease comprising administering an effective amount of a composition comprising a modulator according to claim 1.

18. (canceled)

19. The method according to claim 17 wherein the obesity is high-fat-diet-induced obesity.

20. (canceled)

21. The method according to claim 17 wherein the kidney disease is selected from the group consisting of renal fibrosis, nephrotoxicity, obstructive nephropathy, cisplatin-induced renal disease, acute renal failure, chronic renal failure, renal glomerulopathy and end-stage renal disease.

22. (canceled)

23. The method according to claim 17 wherein the modulator alters the expression level of a cyclophilin D coding region, thereby modulating cyclophilin D biological activity.

24. The method according to claim 17 wherein the modulator decreases cyclophilin D biological activity.

25. The method according to claim 17 wherein the modulator is selected from the group consisting of siRNA, shRNA, miRNA, antisense nucleic acid and triplex-forming nucleic acid.

26. The method according to claim 17 wherein the modulator is selected from the group consisting of an antibody, a peptide, a non-toxic small molecule, cyclosporine A, a cyclosporine A derivative, a cyclosporine A analog, a NICAM and a quinoxaline derivative.

27. The method according to claim 17 wherein the modulator is selected from the group consisting of 2,3-di(furan-2-yl)-6-ethoxycarbonylaminoquinoxaline, 2,3-di(furan-2-yl)-6-N—(N′,N′-diethylcarbamoyl)aminoquinoxaline, 2,3-di(furan-2-yl)-6-((R)-3-ethyoxycarbonyl-piperidino) carbonylaminoquinoxaline, 2,3-di(furan-2-yl)-6-((S)-3-ethyoxycarbonyl-piperidino) carbonylaminoquinoxaline, 2,3-di(furan-2-yl)-6-(pyrrolidin-1-yl) carbonylaminoquinoxaline, 2,3-di(furan-2-yl)-6-morpholinocarbonylaminoquinoxaline, 2,3-di(furan-2-yl)-6-N—(N′,N′-diisopropylcarbamoyl)aminoquinoxaline, Debio 025, N-methyl-4-isoleucine-cyclosporin (NIM811), N-methyl-4-valine-cyclosporin (PKF220-384), Antamanide, SCY-635, a polyketide, 3S-[3R*[E(1S*,3S*,4S*)],4S*,5R*,8S*,9E,12R*,14R*,15S*,16R*,18S*,19S*,26aR*-5,6,8,11,12,13,14,15,16,17,18,19,24,25,26,26a-hexadecahydro-5,19-dihydroxy-3-[2-(4-hydroxy-3-methoxycyclohexyl)-1-methylethenyl]-14,16-dimethoxy-4,10,12,18-tetramethyl-8-(2-propenyl)-15,19-epoxy-3H-pyrido[2,1-c][1,4]oxaazacyclotricosine-1,7,20,21(4H,23H)-tetrone monohydrate (FK-506, tacrolimus), L-685,818 (C18-OH, C21-ethyl-FK-506), NeuroSTAT®, CicloMulsion®, ascomycin (FK-520, L-683,590), [32-Arg] ascomycin, [U-13C] ascomycin, dihydro-FK-506 (FK-506D), C18-OH-ascomycin, A-119,435, U-13C-ascomycin, Leu-Ile,N-(glyoxyl)pipecolate ester, L-732,531, L-688,617, CaN A467-492, mitochondrial-targeted cyclosporine A (mtCsA) and Heat Shock Protein 60.

28. The method according to claim 17 wherein the modulator is cyclosporine A.

29. (canceled)

30. The method according to claim 17 wherein the modulator is in a composition further comprising a second obesity therapeutic selected from the group consisting of zonisamide, topiramate and rimonabant.

31. The method according to claim 17 wherein the modulator is in a composition further comprising a second kidney disease therapeutic selected from the group consisting of Minocycline, Rasagiline, Amifostine, IDN-6556 IDN-6734, VX-740, Adalimumab, Infliximab, Etanercept, ISIS 104828, INO-1001, nicotine amide, Edaravone, Idebenone, CEP-1347 and an apoptosis inhibitor.


SEQUENCE LISTING (2 sequences)

SEQ ID NO: 1 - DNA, 1851 bp, Homo sapiens
  misc_feature (113)..(1222): CypD coding region
ggccggtcagcgtcgctgccggtctccggcggagacggactctggagtttgggcggcccgggcggccactaggtactctgatattccgtactaaacacgtctgcaagtcaagatgtcgcacccgtccccccaagccaagccctccaaccccagtaaccctcgagtcttctttgacgtggacatcggaggggagcgagttggtcgaattgtcttagaattgtttgcagatatcgtacccaaaactgcggaaaattttcgtgcactgtgtacaggagaaaaaggcattggacacacgactgggaaacctctccatttcaaaggatgcccttttcatcgaattattaagaaatttatgattcagggtggagacttctcaaatcagaatgggacaggtggagaaagtatttatggtgaaaaatttgaagatgaaaatttccattacaagcatgatcgggagggtttactgagcatggcaaatgcaggccgcaacacaaacggttctcagttttttatcacaacagttccaactcctcatttggatgggaaacatgtggtgtttggccaagtaattaaaggaataggagtggcaaggatattggaaaatgtggaagtgaaaggtgaaaaacctgctaaattgtgcgttattgcagaatgtggagaattgaaggaaggagatgacgggggaatattcccaaaagatggctctggcgacagtcatccagatttccctgaggatgcggatatagatttaaaagatgtagataaaattttattaataacagaagacttaaaaaacattggaaatacttttttcaaatcccagaactgggagatggctattaaaaaatatgcagaagttttaagatacgtggacagttcaaaggctgttattgagacagcagatagagccaagctgcaacctatagctttaagctgtgtactgaatattggtgcttgtaaactgaagatgtcaaattggcagggagcaattgacagttgtttagaggctcttgaactagacccatcaaataccaaagcattgtaccgcagagctcaaggatggcaaggattaaaagaatatgatcaagcattggctgatcttaagaaagctcaggggatagcaccagaagataaagctatccaggcagaattgctgaaagtcaaacaaaagataaaggcacagaaagataaagagaaggcagtatatgcaaaaatgtttgcttagaaaggattcagttttgcttattgtgtgttgattgtataaatgcaataagaaaatgtaaaggtttttgtctatgaatatgatccctaatgtgtttcttttgacaccttagttccttactgtttacagtttaggagtactgataggggttcatgcttaataaacatgtcacaatacagtaagtaaagtggttttgtttgtttctttgagatggagtcttgctctgtcacccaggctggagtgcggtggcgcaatctcggctcactgcatcctctgcctcccgggttcaagcaattctcctgcctcagcttcccaagtagctgggattacaggcacgtgccaccacgcccagctaatttttgtatttttagtagagatggggtttcaccatattggtcacgtcacgttggtcttgaactcctgaccttgtgatccaccccgccttggcctcccaaagtgctgggattacaggtgtgagccaccgtgcccggccaagtaaaatgttttttaaaatggttatgtgcattattcataaaaaataatggtgtccagtctttttaaacttgtaaagacacatcttattgaataaagagatgagagcttaagtttgtaaaaaaaaaaaaaaaaaa

SEQ ID NO: 2 - protein, 370 aa, Homo sapiens
MSHPSPQAKPSNPSNPRVFFDVDIGGERVGRIVLELFADIVPKTAENFRALCTGEKGIGHTTGKPLHFKGCPFHRIIKKFMIQGGDFSNQNGTGGESIYGEKFEDENFHYKHDREGLLSMANAGRNTNGSQFFITTVPTPHLDGKHVVFGQVIKGIGVARILENVEVKGEKPAKLCVIAECGELKEGDDGGIFPKDGSGDSHPDFPEDADIDLKDVDKILLITEDLKNIGNTFFKSQNWEMAIKKYAEVLRYVDSSKAVIETADRAKLQPIALSCVLNIGACKLKMSNWQGAIDSCLEALELDPSNTKALYRRAQGWQGLKEYDQALADLKKAQGIAPEDKAIQAELLKVKQKIKAQKDKEKAVYAKMFA